(19) United States
(12) Patent Application Publication
(10) Pub. No.: US 2026/0191690 A1
(43) Pub. Date: Jul. 9, 2026

(54) Head-Mounted Electronic Sleep Mask Device with Eye-Responsive Output

(71) Applicants: Andrew Phillip Brimhall, Vineyard, UT (US); Michael George Thatcher, South Jordan, UT (US)

(72) Inventors: Andrew Phillip Brimhall, Vineyard, UT (US); Michael George Thatcher, South Jordan, UT (US)

(21) Appl. No.: 19/433,460

(22) Filed: Dec. 26, 2025

Related U.S. Application Data
(60) Provisional application No. 63/741,845, filed on Jan. 4, 2025.

(51) Int. Cl. A61F 9/04; A61B 3/113; A61M 21/02

(52) U.S. Cl. CPC A61F 9/04; A61B 3/113; A61M 21/02

(57) ABSTRACT

A head-mounted electronic sleep mask device is disclosed that detects eye position and movement of a user's closed eyes and generates responsive output based on the detected eye activity. The device includes a mask portion configured to occlude vision, an eye-tracker sensor comprising one or more emitters configured to emit electromagnetic radiation toward a closed eyelid and one or more photodetectors configured to receive reflected radiation, and a processor in electronic communication with the eye-tracker sensor. The processor analyzes spatial and intensity variations in reflected electromagnetic radiation to generate spatial intensity patterns correlated with eye position beneath the eyelid. Based on the detected eye position or movement, the processor outputs operational commands to control one or more components of the device, including generating audio through one or more speakers or communicating with external devices. The system enables closed-eye, non-visual interaction for sleep assistance and adaptive audio-based experiences.

### CROSS REFERENCE TO RELATED APPLICATIONS

This application claims priority to and the benefit of U.S. Provisional Patent Application No. 63/741,845 filed Jan. 4, 2025 and titled Immersive Sleep System with Non-Visual Interaction Mechanisms and Adaptive Audio-Based Experiences, the entirety of which is incorporated herein by reference.

### BACKGROUND

The present disclosure relates to wearable electronic head-mounted devices, and more particularly to head-mounted electronic sleep mask devices that occlude a user's vision and incorporates eye-tracking sensors and audio output functionality. Various devices and methods have been developed to assist individuals in falling asleep or improving sleep quality. Common approaches include passive and non-electronic sleep masks that block ambient light, white-noise machines, and/or mobile applications that play calming sounds. Existing sleep aids typically lack real-time interaction based on a user's physiological or neurological state during pre-sleep or sleep phases. Conventional eye tracking in virtual or augmented reality systems is designed for visual feedback with eyes open and is not optimized for closed-eye use in low-stimulation environments conducive to sleep. Accordingly, there remains a need for devices that improve a user's ability to fall asleep and that adapt content responsively during pre-sleep and sleep phases.

### SUMMARY

The head-mounted sleep mask device described herein can be worn by a user in pursuit of a goal of obtaining more reliable and consistent sleep. An example head-mounted electronic sleep mask device can include a support portion configured to secure the device to a user's head and a mask portion configured to be placed over the user's eyes to occlude vision during use. The device can include an eye-tracker sensor supported on the mask portion and a processor configured to generate audio or execute commands based on detected eye position and/or movement of a closed eye.

In an example head-mounted electronic sleep mask device, the eye-tracker sensor can include one or more emitters configured to emit electromagnetic radiation (e.g., infrared) toward the eyelid and one or more photodetectors configured to receive reflected radiation and convert it into electrical signals. The processor can analyze spatial and/or intensity variations in the reflected radiation—variations that correlate with eyelid contour changes caused by underlying eye movements—to estimate eye position and detect movement direction.

In an example head-mounted electronic sleep mask device, the processor can output audio signals that cause one or more on-mask speakers to emit audio responsive to detected eye position and/or movement. In an example head-mounted electronic sleep mask device, the processor can execute control commands that cause operations of the head-mounted electronic sleep mask device responsive to detected eye position and/or movement. In an example head-mounted electronic sleep mask device, embodiments, the processor can alternatively, or additionally, output a signal configured to cause an external audio device (e.g., headphones or a speaker) to emit audio responsive to the detected eye position and/or movement.

In an example head-mounted electronic sleep mask device, discrete eye positions can be mapped to distinct audio outputs (e.g., different musical pitches or narrative passages), and detected eye motions and/or directions of view can modulate ongoing audio (e.g., pitch or timbre changes). In an example head-mounted electronic sleep mask device, discrete eye positions can additionally or alternatively be mapped to control commands of the head-mounted electronic sleep mask device. In an example head-mounted electronic sleep mask device, a content engine stored in computer memory can render narrative, melodic, and task-guidance content. In another example, a spatial-audio engine can place or orient virtual sound sources and simulated objects in a perceived acoustic scene. A software program stored in computer memory (e.g., a non-transitory computer-readable storage medium) and executed by the processor can detect a user approaching sleep and can adapt pacing and volume of audio feedback as the user approaches sleep.

These and other example embodiments are described in further detail below with reference to the drawings.

### BRIEF DESCRIPTION OF THE DRAWINGS

FIG. 1 is a front perspective view of an exemplary head-mounted electronic sleep mask device being worn by a user.

FIG. 2 is a front perspective view of an exemplary head-mounted electronic sleep mask device being located off of the user and showing an exterior facing side of the sleep mask device configured to face away from the user when in use.

FIG. 3 is a rear perspective view of an exemplary head-mounted electronic sleep mask device, showing an interior facing side of the sleep mask device configured to face the eyes of a user when in use.

FIG. 4 is an exploded assembly view of an exemplary head-mounted electronic sleep mask device.

FIG. 5 is a perspective view of an exemplary right eye sensor of an eye tracker sensor of an exemplary head-mounted electronic sleep mask device.

FIG. 6 is a schematic diagram of an exemplary head-mounted electronic sleep mask device according to at least one example of the disclosure.

FIG. 7 is a schematic diagram of an exemplary computing device of an exemplary head-mounted electronic sleep mask device according to at least one example of the disclosure.

FIGS. 8A-8C are depictions of closed-eye reflectance sensing, showing emitter illumination of the eyelid, photodetector reception, and example sensor outputs in which spatial intensity varies with left, center, and right closed-eye positions.

FIG. 9 is an illustration of detecting closed eye position of the eyes of a user based on sensor output and spatial intensity patterns detected by a photodetector and analyzed by a processor.

FIG. 10 is an illustration of exemplary sensor outputs and spatial intensity patterns detected by a photodetector, analyzed by a processor, and associated with closed-eye positions of eyes of a user of an exemplary head-mounted electronic sleep mask device.

FIG. 11 is an illustration of closed-eye movement over a certain time period and sensor output and spatial intensity patterns produced therefrom.

FIG. 12 is a schematic diagram of an exemplary head-mounted electronic sleep mask device in connection with external devices, according to at least one example of the present disclosure.

FIG. 13 is an exemplary method of determining closed eye positions of a user using an exemplary head-mounted electronic sleep mask device according to at least one example of the present disclosure.

FIG. 14 is a schematic diagram of closed eye position profiles saved in memory devices and associated with preset operational commands of the sleep mask device.

FIG. 15 is an exemplary method of determining closed eye positions of a user using an exemplary head-mounted electronic sleep mask device according to at least one example of the present disclosure.

FIG. 16 is an exemplary method of determining closed eye positions of a user using an exemplary head-mounted electronic sleep mask device according to at least one example of the present disclosure.

FIG. 17 is a flowchart diagram of exemplary workflow of an exemplary head-mounted electronic sleep mask device according to at least one example of the present disclosure.

### DETAILED DESCRIPTION

While these exemplary embodiments are described in sufficient detail to enable those skilled in the art to practice the invention, it should be understood that other embodiments may be realized and that various changes to the invention may be made without departing from the spirit and scope of the present invention. Thus, the following more detailed description of the embodiments of the present invention is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

### Definitions

In describing and claiming the present invention, the following terminology will be used.

The singular forms “a,” “an,” and “the” include plural referents unless the context clearly dictates otherwise. Thus, for example, reference to “a species” includes reference to one or more of such materials and reference to “introducing” refers to one or more of such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, the term “about” is used to provide flexibility and imprecision associated with a given term, metric or value. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, a plurality of items, structural elements, compositional elements, and/or materials may be presented in a common list for convenience. However, these lists should be construed as though each member of the list is individually identified as a separate and unique member. Thus, no individual member of such a list should be construed as a de facto equivalent of any other member of the same list solely based on their presentation in a common group without indications to the contrary.

As used herein, the term “at least one of” is intended to be synonymous with “one or more of.” For example, “at least one of A, B and C” and “at least one of A, B or C” explicitly includes only A, only B, only C, or combinations of each.

Numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

As used herein, “substantially similar” refers to a degree of similarity between two signals, patterns, or data representations that is sufficient for a processor to determine that the signals, patterns, or data representations correspond to the same underlying eye position or eye movement, notwithstanding variations caused by noise, sensor placement, user physiology, device fit, or environmental conditions.

### Head-Mounted Electronic Sleep Mask Device

The embodiments described herein relate to head-mounted, electronic sleep mask devices that occlude a user's vision and generate audio, and/or perform control commands and operations of the sleep mask device, in response to eye position or eye movement detected while a user's eyes are closed. The systems and methods provided herein allow for providing a user with activities, actions, and stimulation meant to more effectively lull a user to sleep. The exemplary embodiments described herein, allow for stimulation and feedback to be provided to a user whose eyes are closed, thereby being more conducive to helping a user wearing the device to fall asleep. The following description presents exemplary embodiments, structures, methods, and operations that support the claims. Similar reference numerals denote similar elements throughout the figures.

In at least one example, the systems, methods, and devices described herein can include and/or utilize a head-mounted electronic sleep mask device, which for convenience, is also referred to herein as a sleep mask device. The head-mounted electronic sleep mask device can include a support portion configured to secure the sleep mask device to a head of a user when in use. The head-mounted electronic sleep mask device can further include a mask portion connected to the support portion and configured to be placed over one or more eyes of a user to occlude vision of the user when in use. The head-mounted electronic sleep mask device can further include an eye tracker sensor supported on the mask portion and configured to output an eye position signal indicating an eye position of one or more eyes of the user. A processor in electronic communication with the eye-tracker sensor and the one or more speakers can further be included on the head-mounted electronic sleep mask device. A non-transitory computer-readable storage medium can be included in at least one example and can store instructions that, when executed by the processor, executes an operational command of the head-mounted electronic sleep mask device. The operational command executed by the processor based on the instructions is based on an eye position detected based on the eye position signal output by the eye tracker sensor. More particularly, detection of the eye position beneath the eyelid is based on the eye position signal output by the eye tracker sensor 140 and detected based on the analysis of the processor performed on the eye position signal.

The head-mounted electronic sleep mask device can further include one or more speakers configured to output audio. A processor in electronic communication with the eye-tracker sensor and the one or more speakers can further be included on the head-mounted electronic sleep mask device. A non-transitory computer-readable storage medium can be included in at least one example and can store instructions that, when executed by the processor, cause the processor to output an audio signal configured to cause the one or more speakers to output an audio sound. The audio signal output by the processor to cause the speaker to output the audio sound is based on the eye position signal output by the eye tracker sensor.

In some examples, the head-mounted electronic sleep mask device can include an eye tracker sensor that is configured to produce the eye position signal, indicating the eye position of the one or more eyes, when the one or more eyes are closed based on the shape of an eyelid covering the eye.

In some examples, the eye-tracker sensor can include one or more emitters configured to emit electromagnetic radiation. The eye-tracker sensor can further include one or more photodetectors configured to convert electromagnetic radiation emitted by the emitters to the eye position signal. The eye position signal can be output to the processor for analysis.

In some examples, when the head-mounted electronic sleep mask device is in use, the electromagnetic radiation from the one or more emitters is incident on a portion of the eyelid covering the eye and the one or more photodetectors receives the electromagnetic radiation from the one or more emitters and converts the electromagnetic radiation to the eye position signal indicating a position of the eye based on the contour of the eyelid covering the eye of the user.

In some examples, when the head-mounted electronic sleep mask device is in use, the electromagnetic radiation from the one or more emitters is incident on a portion of the eyelid covering the eye and the one or more photodetectors receive reflected electromagnetic radiation from the external surface of the eyelid and produce the eye position signal, indicating the position of the eye, based on one or more of spatial and intensity variations in the reflected electromagnetic radiation from the external surface of the eyelid, wherein the spatial and intensity variations correlate with eye positions and/or underlying eye movements of the eye of the user.

In some examples of the head-mounted electronic sleep mask device, the processor can output a first audio signal to the one or more speakers causing the one or more speakers to output a first audio sound when the eye position signal from the eye tracker sensor indicates the position of the one or more eyes of the user is at a first position.

In some examples of the head-mounted electronic sleep mask device, the processor can output a second audio signal to the one or more speakers causing the one or more speakers to output a second audio sound different from the first audio sound when the eye position signal from the eye tracker sensor indicates the position of the one or more eyes of the user is at a second position different than the first position.

In some examples of the head-mounted electronic sleep mask device, the processor can output a third audio signal to the one or more speakers causing the one or more speakers to output a third audio sound when the eye position signal from the eye tracker sensor indicates motion of the eyes of the user in a first direction.

In some examples of the head-mounted electronic sleep mask device, the processor can output a fourth audio signal to the one or more speakers causing the one or more speakers to output a fourth audio sound different from the third audio sound when the eye position signal from the eye tracker sensor indicates motion of the eyes of the user in a second direction different from the first direction.

In some examples of the head-mounted electronic sleep mask device, the first audio sound can be a first musical pitch and the second audio sound can be a second musical pitch different from the first musical pitch.

In some examples of the head-mounted electronic sleep mask device, the third audio sound and the fourth audio sound can each be modulations of one or more of the first audio sound and the second audio sound.

In some examples of the head-mounted electronic sleep mask device, the first audio sound can be a first narrative passage and the second audio sound can be a second narrative passage different from the first narrative passage.

In some examples, when the head-mounted electronic sleep mask device is in use, the processor can output audio signals to the one or more speakers causing the one or more speakers to output audio sounds of a narrative to the user. The processor can further output audio signals to the one or more speakers to alter the narrative based on one or more of the position and movement/motion of the eye beneath the eyelid detected based on the eye position signal output by the eye tracker sensor.

In some examples, when the head-mounted electronic sleep mask device is in use, the processor can output audio signals to the one or more speakers causing the one or more speakers to output audio sounds of a melody to the user. The processor can output audio signals to the one or more speakers to alter the melody based on one or more of a position and a movement of the eye beneath the eyelid detected based on the eye position signal output by the eye tracker sensor.

In some examples, when the head-mounted electronic sleep mask device is in use, the processor can output audio signals to the one or more speakers causing the one or more speakers to output audio sounds of instructions to perform tasks to the user. The processor can output audio signals to the one or more speakers to indicate a status of the tasks being performed by the user based on one or more of a position and a motion of the eye beneath the eyelid detected based on the eye position signal output by the eye tracker sensor.

In some examples, when the head-mounted electronic sleep mask device is in use, the processor can output audio signals to the one or more speakers causing the one or more speakers to output audio sounds to create a spatial audio environment. The processor can output audio signals to the one or more speakers to alter an orientation of the spatial audio environment based on one or more of a position and a motion of the eye beneath the eyelid detected based on the eye position signal output by the eye tracker sensor.

In some examples, when the head-mounted electronic sleep mask device is in use, the processor can output audio signals to the one or more speakers, causing the one or more speakers to output audio sounds simulating a presence of one or more simulated objects in the spatial audio environment. The processor can output audio signals to the one or more speakers to simulate user interaction with the one or more simulated objects in the spatial audio environment based on one or more of a position and a motion of the eye beneath the eyelid detected based on the eye position signal output by the eye tracker sensor.

In some examples, when the head-mounted electronic sleep mask device is in use, the processor can output a first audio signal to the one or more speakers causing the one or more speakers to output a first audio sound when the eye-tracker sensor detects motion of the eyes of the user in a first direction.

In some examples, when the head-mounted electronic sleep mask device is in use, the processor can output a second audio signal to the one or more speakers causing the one or more speakers to output a second audio sound different from the first audio sound when the eye-tracker sensor detects motion of the eyes of the user in a second direction different from the first direction.

In at least one example, of the head-mounted electronic sleep mask device, the eye-tracker sensor can include a right-eye sensor configured to track a position of a right eye of the user. The right-eye sensor can include one or more right-eye emitters configured to emit electromagnetic radiation toward the right eye of the user and one or more right-eye photodetectors configured to convert the electromagnetic radiation to a right-eye position signal that is output from the right-eye photodetector to the processor. The eye-tracker sensor can further include a left-eye sensor configured to track a position of a left eye of the user. The left-eye sensor can include one or more left-eye emitters configured to emit electromagnetic radiation toward the left eye of the user and one or more left-eye photodetectors configured to convert the electromagnetic radiation to a left-eye position signal that is output from the left-eye photodetector to the processor. In at least one example, the systems, methods, and devices described herein can include and/or utilize a head-mounted electronic sleep mask device. The head-mounted electronic sleep mask device can include a support portion configured to secure the sleep mask device to a head of a user when in use. The head-mounted electronic sleep mask device can further include a mask portion connected to the support portion and configured to be placed over eyes of a user to occlude vision of the user when in use. The head-mounted electronic sleep mask device can further include an eye-tracker sensor supported on the mask portion and configured to produce an eye position signal indicating an eye position of one or more eyes of the user. The head-mounted electronic sleep mask device can further include a processor in electronic communication with the eye-tracker sensor. The head-mounted electronic sleep mask device can further include a non-transitory computer-readable storage medium that stores instructions that, when executed by the processor, cause the processor to output a signal configured to cause a speaker to output an audio sound. The signal output by the processor is based on the eye position signal, indicating the eye position of the one or more eyes of the user, that is output by the eye-tracker sensor. The speaker can be a speaker external to the head-mounted electronic sleep mask device.

In at least one example, the systems, methods, and devices described herein can include and/or utilize a head-mounted electronic sleep mask device. The head-mounted electronic sleep mask device can include a support portion configured to secure the sleep mask device to a head of a user when in use. The head-mounted electronic sleep mask device can further include a mask portion connected to the support portion and configured to be placed over eyes of a user to occlude vision of the user when in use. The head-mounted electronic sleep mask device can further include an eye-tracker sensor supported on the mask portion and configured to produce an eye position signal indicating an eye position of one or more eyes of the user. The head-mounted electronic sleep mask device can further include a processor in electronic communication with the eye-tracker sensor. The head-mounted electronic sleep mask device can further include a non-transitory computer-readable storage medium that stores instructions that, when executed by the processor, executes an operational command of the head-mounted electronic sleep mask device. In at least one example, the operational commend executed by the processor based on the instructions is based on the eye position signal output by the eye tracker sensor.

### Sleep Mask Device Architecture

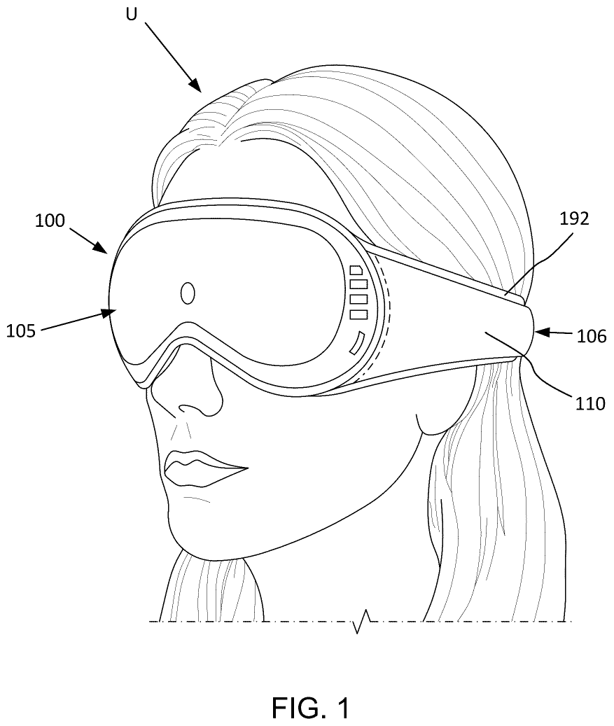

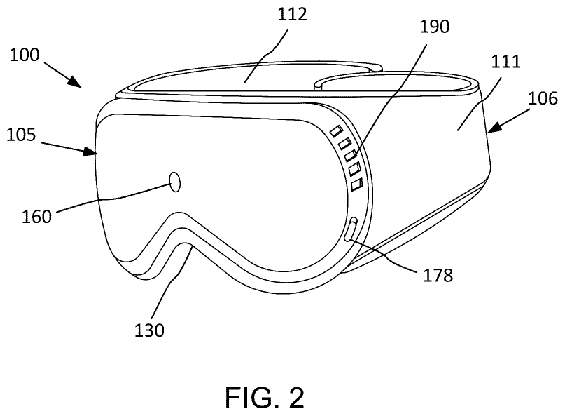

Turning now to the drawings, FIG. 1 shows a front perspective view of an example head-mounted, electronic, sleep mask device 100 being worn by a user U. For convenience, the head-mounted, electronic, sleep mask device will hereinafter be referred to in a shortened manner simply as sleep mask device. Sleep mask device 100 is a head-mounted, electronic sleep mask device in accordance with at least one example of the present disclosure. As shown in FIG. 1, sleep mask device 100 is wearable by a user U. Sleep mask device 100 includes a mask portion 105 and a support portion 106. Sleep mask device 100 is shown worn on user U's head and face in a position where mask portion 105 is covering the eyes of user U to occlude the vision of user U. Mask portion 105 will be described in further detail with reference to other FIGS. 2-4. Support portion 106 is connected to mask portion 105 and is configured to secure sleep mask device 100 to a head of user U. More specifically, support portion 106 includes an adjustable strap 110 configured to secure sleep mask device 100 to user U, specifically on user U's head. A cover 192, made of fabric or other soft material, covers adjustable strap 110 to add to the comfort and wearability of sleep mask device 100.

The adjustable strap 110 can be configured to maintain sleep mask device 100 in a comfortable and stable position during use, including while the user is standing, sitting, asleep, or reclining. The methods, structures, and configuration of adjustable strap 110 to adjust to the size of user U's head are not intended to be particularly limited by this disclosure and can be accomplished by diverse configurations and methods and structures. For example, adjustable strap 110 can be implemented in numerous configurations, and the following examples are provided for illustrative purposes and should not be viewed as limiting. For example, the adjustable strap 110 may include one or more flexible or semi-rigid bands extending around or over portions of the user's head to support the mask body. The material of adjustable strap 110 and/or cover 192 may include soft fabric, elastic webbing, breathable mesh, microfiber, silicone, foam-lined polymer, or composite materials chosen to reduce skin irritation, noise, and pressure points during extended wear. The length of adjustable strap 110 or tension thereof may be varied using a mechanical, elastic, or electromechanical adjustment feature, which may include, without limitation, hook-and-loop fasteners, sliding buckles or friction clamps that allow incremental tightening or loosening, dial-based or ratcheting tensioners allowing fine adjustment with tactile feedback, elastic expansion zones or self-tensioning segments providing adaptive fit as the user moves during sleep, magnetic coupling elements configured for quick detachment and silent reconnection, telescoping or rail-based members incorporated within the side or top straps to permit controlled movement along an axis, drawstring or cord-lock systems with smooth adjustment suitable for low-profile integration, spring-biased or pneumatic tensioners providing self-regulating strap tension, inflatable or air-cell cushions that can be inflated or deflated to modify strap pressure or contour, or shape-memory or thermally responsive materials that adapt to the head contour when activated by body heat or a low-voltage heating element.

In certain embodiments, adjustable strap 110 may include multiple segments such as a rear occipital band, top stabilizing strap, and one or more side connector portions, each of which can be individually adjustable to distribute load evenly. Adjustment may be manual, automated, or motor-assisted, such as through compact micro-actuators or servo-driven spools controlled by the device's onboard electronics. Adjustable strap 110 may also include sensors (e.g., tension sensors, pressure sensors, or proximity detectors) configured to monitor fit and comfort parameters. These sensors may communicate with the mask's control circuitry to automatically adjust strap tension in response to user position, detected movement, or biometric data (e.g., heart rate or sleep stage), thereby maintaining comfort without user intervention. In some embodiments, adjustable strap 110 may incorporate integrated wiring channels or flexible printed circuits allowing power and data connections to pass between strap sections and the mask body while maintaining a smooth, low-profile surface. Padding or cover materials may be removable or washable, and adjustable strap elements may be interchangeable to accommodate different head sizes or user preferences.

The described features may be combined or omitted in any suitable manner. Unless expressly stated otherwise, the examples provided herein are non-limiting and intended to illustrate representative ways in which an adjustable strap may be configured to support a head-mounted electronic sleep mask device.

FIG. 2 is a front perspective view of sleep mask device 100 being located off of a user and showing an exterior facing side of sleep mask device 100 configured to face away from the user when in use. FIG. 2 shows an outer surface 111 of sleep mask device 100. As will be appreciated viewing FIG. 2 in conjunction with FIG. 1, outer surface 111 is configured to face outward and away from the user when sleep mask device 100 is in use, and/or being worn on the head of a user, such as user U. FIG. 2 further shows an inner surface 112 that is configured to face inward toward the user when sleep mask device 100 is in use, and/or being worn on the head of a user.

Mask portion 105 further includes an outer shell 102 configured to house and/or support various components of sleep mask device 100. As shown in FIG. 2, mask portion 105 can include a microphone 160 supported on mask portion 105. Microphone 160 is shown substantially in the center of mask portion 105 and outer shell 102. However, it is to be understood that multiple configurations, placements, and numbers of microphones can be achieved for the sleep mask device and are considered to be within the scope of this disclosure. For example, multiple microphones can be used, substantially located on the left and right of mask portion 105 or outer shell 102 to achieve stereo recording of sounds. Left and right microphones can be used in addition to microphone 160 or alternatively to microphone 160. Any number of microphones on mask portion 105 can be used and any location desired can be used for placement on the sleep mask 100, including, if desired, placement of microphones on support portion 160.

As further shown in FIG. 2, openings 190 can be formed in outer shell 102 to allow for ventilation and airflow to flow to and from a user's face to improve comfort, useability, and wearability for users of sleep mask device 100. FIG. 2 shows multiple openings formed in outer shell 102. However, it is to be understood that any number of openings, including one opening or more openings can be utilized to facilitate ventilation for sleep mask device 100.

As further shown in FIG. 2, an electronic interface 178 is formed in outer shell 102. Electronic interface 178 can be an electrical port configured to receive an electronic connector and to facilitate power delivery to sleep mask device 100. Electronic interface 178 can be in wired connection with a rechargeable battery supported on sleep mask device 100 to allow for wired charging of sleep mask device 100. Electronic interface can further or alternatively be configured to facilitate wireless charging of sleep mask device 100. Electronic interface 178 can further be a port configured to receive an electronic connector that delivers direct electrical connection to a power source such as an external battery, solar power, or a power socket in electrical communication with a power grid. The power source used to power sleep mask device 100 is not intended to be particularly limited by this disclosure.

FIG. 2 further shows an external light seal 130 supported by outer shell 102 of mask portion 105, at a position that is configured to rest on a nose of a user when sleep mask device 100 is in use or being worn by a user. External light seal 130 can be made of a compliant material that deforms and forms to at least a portion of the face of a user to reduce stray light from an environment external to sleep mask device 100 to block out ambient light and to create an experience for the user that is conducive to sleeping.

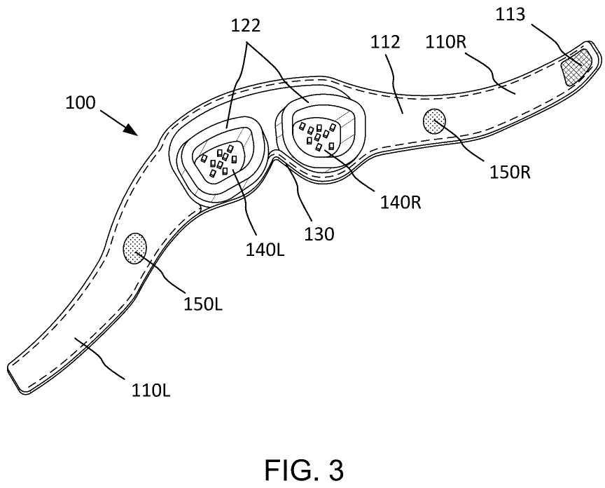

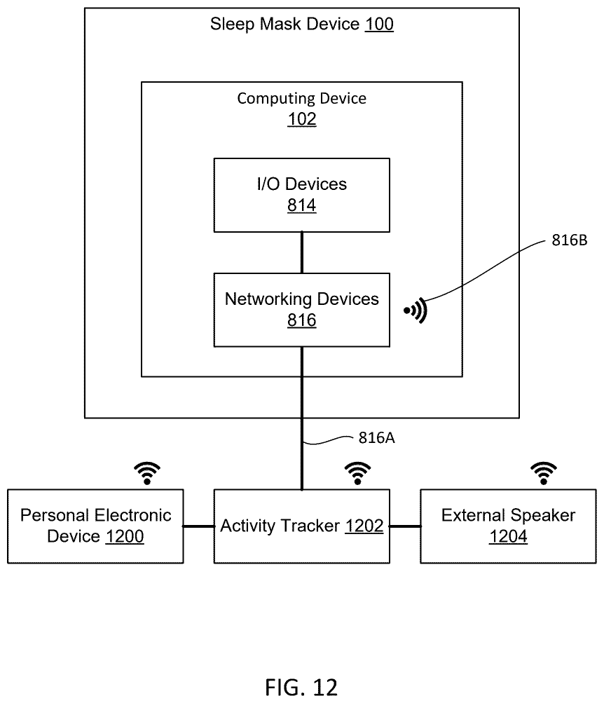

FIG. 3 shows a rear perspective view of sleep mask device 100, showing an interior facing side of the sleep mask device configured to face the eyes of a user when in use. In other words, the view of sleep mask device 100 shown in FIG. 3 shows a view of inner surface 112 of sleep mask device 100 that is configured to face inward towards a user's head and face when sleep mask device 100 is in use and/or being worn by a user, such as user U. Sleep mask device 100 can further include one or more speakers 150 configured to provide audio to a user. As shown, a pair of speakers 150L and 150R can be used with a speaker 150R on the right side configured to be placed on or near a user's right ear and a speaker 150L on the left side configured to be placed on or near a user's left ear to facilitate stereo sound being provided to the user. The manner and types of sounds, audio, and feedback that can be provided to the user through speakers 150 are not particularly limited by this disclosure. Audio output may be provided to on-mask speakers 150L/150R alone. Alternatively or in addition, audio may be output via a wireless interface to an external device (e.g., personal electronic device 1200, activity tracker 1202, and/or external speaker 1204 of FIG. 12) running a companion application or to external speakers or headphones.

Specific examples of audio feedback will be described in further detail with reference to other figures.

FIG. 3 further shows that adjustable strap 110, according to at least one example, includes a left strap 110L and a right strap 110R. At least one of the straps of left strap 110L and right strap 110R (e.g. the right strap 110R) includes an anchor 113 that can be used to attach one strap (e.g. right strap 110R) to the other (e.g. left strap 110L). Anchor 113 can be hook and loop, a fastener, a button, or any other method of adjusting and fitting adjustable strap 110 to the head of a user. The methods of sizing, attaching, fitting, and wearing the adjustable strap are not intended to be particularly limited by this disclosure.

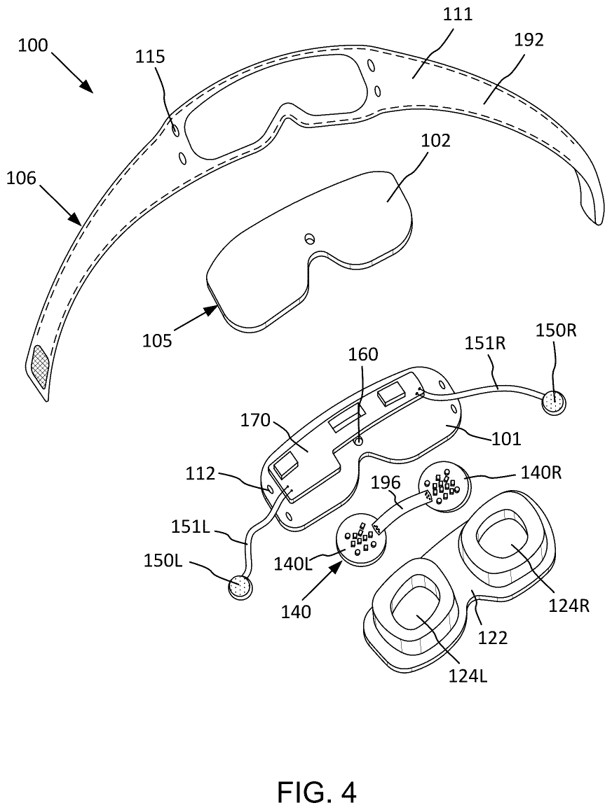

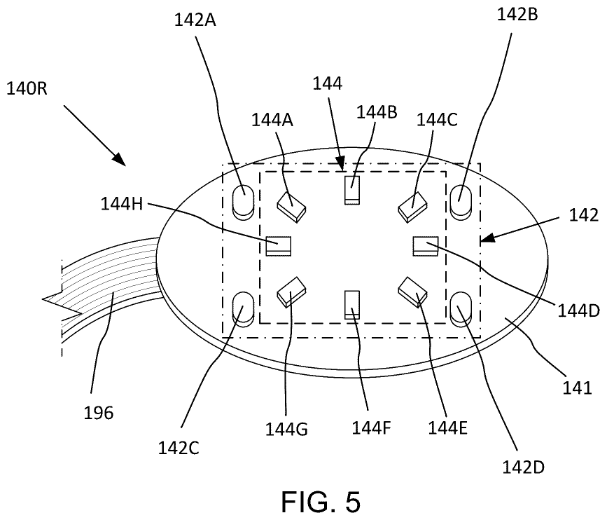

FIG. 3 further shows a compliant, face-contacting foam gasket 122 that is configured to contact an eye region of the user when sleep mask device 100 is being worn and/or is being used by a user, such as user U. Foam gasket 122 can be made of a soft, pliable, and/or compliant material configured to deform and form to the shape of a user's face when the mask is worn by the user in order to ensure a comfortable experience for a user, and to block out light from the users eyes when wearing sleep mask device 100 to create and experience for the user conducive to allowing the user to fall asleep. An eye tracker sensor 140, including a right eye tracker sensor 140R and a left eye tracker sensor 140L, can be disposed within the foam gasket 122. The function and configuration of the eye tracker sensor 140 will be described in further detail with reference to FIGS. 4 and 5 in this disclosure.

FIG. 4 is an exploded assembly view of sleep mask device 100. As shown, sleep mask device 100 includes a fabric cover 192 that can be attached to outer shell 102. Sleep mask device 100 further includes internal frame 101. One or more attachment points 112 are formed on internal frame 101 and are configured to attach the internal frame 101 to fabric cover 192 at one or more cover attachment points 115. The attachment points 112 and cover attachment points 115 are not particularly limited by this disclosure. Any suitable form of attaching a fabric cover 192 to internal frame 101 can be used, for example, hook and loop, button and hole, ties, laces, adhesive, thread, or any other suitable method of attachment can be used to attach internal frame 101 to fabric cover 192.

Internal frame 101 further supports an electronic substrate 170. Electronic substrate 170 may be one or more substrates configured to support and electrically interconnect a plurality of electrical components. Electronic substrate 170 may serve as a physical platform upon which integrated circuits, passive components, interconnect structures, or other electrical elements are mounted, embedded, or otherwise disposed. Electronic substrate 170 may take a variety of forms and may be composed of any suitable material or combination of materials that provides mechanical support and enables electrical communication between components.

Exemplary substrates include, but are not limited to: printed circuit boards (PCBs); interposers; wafers or wafer-level packages; ceramic substrates; flexible or rigid-flex circuit boards; semiconductor substrates; polymer-based or organic laminates; glass or sapphire-based substrates; and any other structure capable of supporting conductive traces, vias, pads, or other electrical interconnection features. In certain embodiments, electronic substrate 170 may be multilayered, comprising one or more dielectric layers interleaved with conductive layers to form internal routing structures, power and ground planes, or signal traces.

Electrical communication between components disposed on electronic substrate 170 may be facilitated through conductive features such as traces, vias, through-holes, microvias, redistribution layers, or solder bumps. These interconnection features may be formed using conductive materials such as copper, gold, aluminum, tungsten, or other suitable metals or alloys. In some embodiments, electronic substrate 170 may further include embedded components, surface-mounted components, or integrated passive or active devices that contribute to signal conditioning, impedance control, or power distribution. Electronic substrate 170 may be configured to support high-frequency or high-speed data communication, power delivery, or mixed-signal operation. It may also be adapted for use in a variety of form factors, including planar, stacked, folded, or three-dimensional configurations. Further, electronic substrate 170 may be implemented as part of a system-in-package (SiP), multi-chip module (MCM), or other integrated assembly where multiple dies or components are electrically coupled through the electronic substrate 170.

Unless explicitly stated otherwise, references to a “electronic substrate” herein should be understood to include any structure capable of physically supporting and electrically interconnecting circuit elements. The selection of substrate type, material composition, thickness, and electrical interconnect structure may be determined according to desired electrical, thermal, mechanical, or manufacturing characteristics.

Various electronic components are supported on, and/or are in electrical communication with electronic substrate 170 to facilitate communication between the electronic components and proper operation of the mask. For example, one or more components of a processor, a memory, a rechargeable battery, a wired and/or wireless charging interface, a wireless communication interface, speaker, eye-tracker sensor, microphone, cameras, are supported on and/or in electrical communication with electronic substrate 170 and other electrical components supported thereon or connected thereto.

Various electronic components are included in sleep mask device 100 that are in electronic communication with electronic substrate 170 and configured to operate, via signals based on the instructions stored in computer-readable memory, executed by a processor, and relayed via electronic pathways on electronic substrate 170, or wirelessly via a wireless communication interface. Microphone 160 is shown supported on internal frame 101. Speakers 150L and 150R, are connected to electronic substrate 170 via wired connections 151L and 151R. Although microphone 160 and speakers 150L and 150R are shown in connection with and/or wired to electronic substrate 170, it is to be understood that either of microphone 160 and speakers 150L and 150R can be connected to sleep mask device 100 via Bluetooth or other wireless communication methods without departing from the scope of this disclosure. In other words, microphone 160 and speakers 150L and 150R, are in electrical communication with electronic substrate 170 and configured to operate, via signals based on the instructions stored in computer-readable memory, executed by a processor, and relayed via electronic pathways on the electronic substrate 170 or wirelessly via a wireless communication interface. A left speaker 150L is placed on the sleep mask device 100 at a position corresponding to a left ear of a user when sleep mask device 100 is being worn by the user. Left speaker 150L is configured to deliver sound to a left ear of a user. A right speaker 150R is placed on the sleep mask device 100 at a position corresponding to a right ear of a user when sleep mask device 100 is being worn by the user. Right speaker 150R is configured to deliver sound to a right ear of a user.

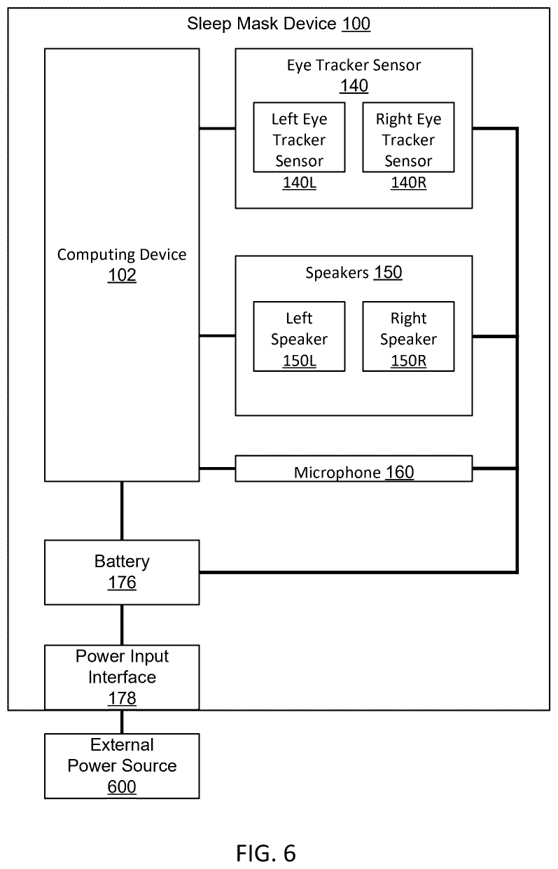

Sleep mask device 100 includes an eye-tracker sensor 140 in electrical communication with the electronic substrate 170. By being in electrical communication with electronic substrate 170, eye-tracker sensor 140 is also in electrical communication with a processor, computer-readable memory, rechargeable battery, wireless communication interface, each of which are described later with reference to FIG. 6. Eye-tracker sensor 140 is further in electrical communication with, microphone 160 and speakers 150L and 150R via electronic substrate 170, and any other components in electrical communication with electronic substrate 170. Accordingly, eye movements detected by the eye-tracker sensor can be used as inputs that alter outputs by sleep mask device 100. Instructions stored in a computer-readable memory and executed by a processor can be triggered, altered, interacted with, or otherwise affected by eye movements of a user wearing sleep mask device 100, whose eye movements are tracked using eye-tracker sensor 140.

Eye-tracker sensor 140 can include a left-eye sensor 140L and a right-eye sensor 140R. The left-eye sensor 140L is positioned on the sleep mask device 100 at a position corresponding to the left eye of the user when sleep mask device 100 is worn by the user. The left-eye sensor 140L is positioned to be configured to cover the left eye of the user when sleep mask device 100 is in use. The right-eye sensor 140R is positioned on the sleep mask device 100 at a position corresponding to the right eye of the user when sleep mask device 100 is worn by the user. The right-eye sensor 140R is positioned to be configured to cover the right eye of the user when sleep mask device 100 is in use. A flexible interconnect 196 is shown connecting right-eye sensor 140R and left-eye sensor 140L. Flexible interconnect 196 can be a connection comprising wire, fiber optic, or any other suitable connector for providing electrical or other types of data signals between right-eye sensor 140R and left-eye sensor 140L.

While not shown, it is to be understood that flexible interconnect 196 can be omitted and the right-eye sensor 140R can be in electrical communication with electronic substrate 170 such that electrical signals, electrical power, and/or other types of data signals, can be relayed between right-eye sensor 140R and the processor and computer-readable memory via electronic substrate 170. Similarly, the left-eye sensor 140L can be in electrical communication with electronic substrate 170 such that electrical signals, electrical power, and/or other types of data signals, can be relayed between left-eye sensor 140L, the processor, and the computer-readable memory via electronic substrate 170. In other words, left-eye sensor 140L and right-eye sensor 140R can communicate with each other, the processor, and the computer-readable memory via electrical pathways on electronic substrate 170, or via a flexible interconnect that connects left-eye sensor 140L and right-eye sensor 140R to each other.

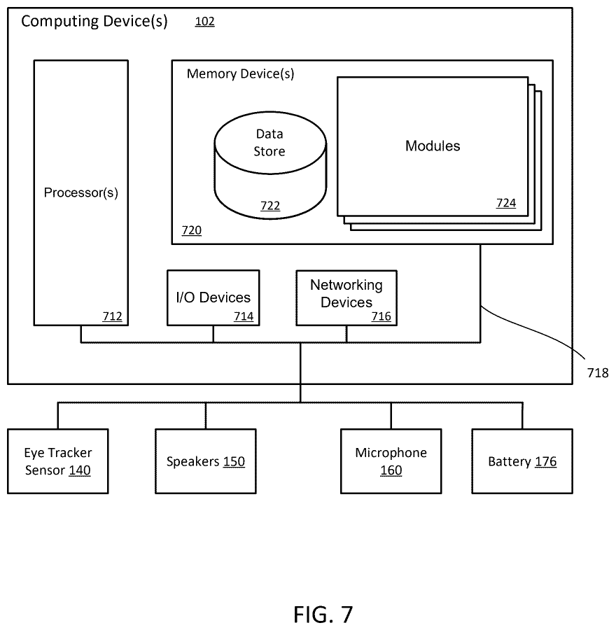

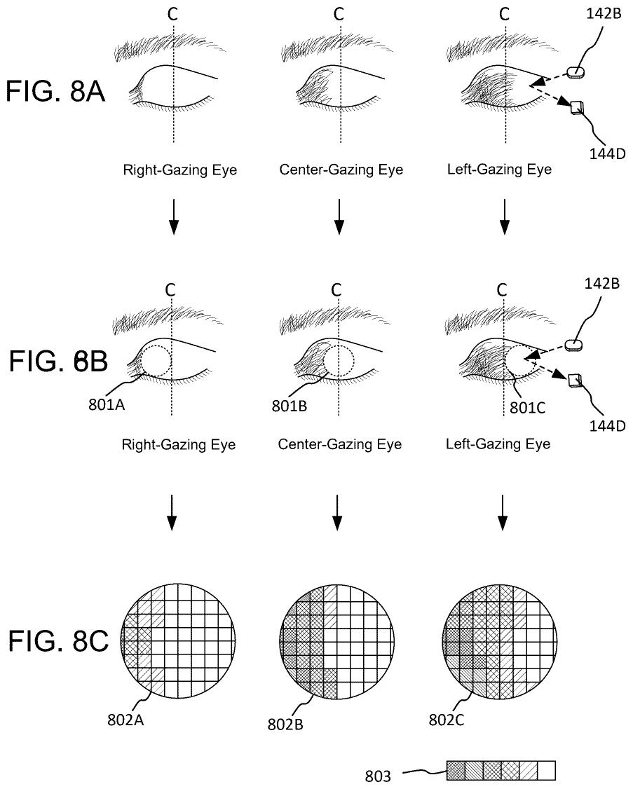

The various electronic components of the sleep mask device 100, as well as communication and operation of the electronic components will be described later with reference to FIGS. 7 and 8.

As described earlier, and as shown in FIG. 4, sleep mask device 100 includes a compliant face-contact foam gasket 122 configured to cover the user's eyes and occlude vision during use. External light seal 130 reduces stray light from entering the mask at a region near the user's nose. Compliant face-contact foam gasket 122 can define dome-shaped, eye-relief regions 124L and 124R for the left and right eyes, respectively, to allow a user's eyes to have space to move freely within the sleep mask device 100. Free movement of eyes within the mask can allow for a user to direct their eyes and line of sight in any direction without being blocked by materials, components, or structures of sleep mask device 100.

FIG. 5 is a perspective view of right-eye sensor 140R of eye tracker sensor 140 of sleep mask device 100. While only right-eye sensor 140R is shown, it is to be understood that the configuration of left-eye sensor 140L will be substantially similar to that of right-eye sensor 140R, including emitters, photodetectors, and a substrate in substantially the same arrangement as shown for right-eye sensor 140R. As such, the description of FIG. 4 with reference to the right-eye sensor 140R applies as well to left-eye sensor 140R. As shown, right-eye sensor 140R includes a substrate 141 that facilitates electronic communication between electrical components supported thereon and connected thereto. Substrate 141 may be one or more substrates configured to support and electrically interconnect a plurality of electrical components. Substrate 141 may serve as a physical platform upon which integrated circuits, passive components, interconnect structures, or other electrical elements are mounted, embedded, or otherwise disposed. Substrate 141 may take a variety of forms and may be composed of any suitable material or combination of materials that provides mechanical support and enables electrical communication between components.

Exemplary substrates include, but are not limited to: printed circuit boards (PCBs); interposers; wafers or wafer-level packages; ceramic substrates; flexible or rigid-flex circuit boards; semiconductor substrates; polymer-based or organic laminates; glass or sapphire-based substrates; and any other structure capable of supporting conductive traces, vias, pads, or other electrical interconnection features. In certain embodiments, the substrate 141 may be multilayered, comprising one or more dielectric layers interleaved with conductive layers to form internal routing structures, power and ground planes, or signal traces.

Electrical communication between components disposed on the substrate 141 may be facilitated through conductive features such as traces, vias, through-holes, microvias, redistribution layers, or solder bumps. These interconnection features may be formed using conductive materials such as copper, gold, aluminum, tungsten, or other suitable metals or alloys. In some embodiments, the substrate 141 may further include embedded components, surface-mounted components, or integrated passive or active devices that contribute to signal conditioning, impedance control, or power distribution. The substrate 141 may be configured to support high-frequency or high-speed data communication, power delivery, or mixed-signal operation. It may also be adapted for use in a variety of form factors, including planar, stacked, folded, or three-dimensional configurations. Further, the substrate 141 may be implemented as part of a system-in-package (SiP), multi-chip module (MCM), or other integrated assembly where multiple dies or components are electrically coupled through the substrate 141.

Unless explicitly stated otherwise, references to a “substrate” herein should be understood to include any structure capable of physically supporting and electrically interconnecting circuit elements. The selection of substrate type, material composition, thickness, and electrical interconnect structure may be determined according to desired electrical, thermal, mechanical, or manufacturing characteristics.

Referring back to FIGS. 3 and 4, right-eye sensor 140R and left-eye sensor 140L are disposed to align with eye-relief region 124 defined in face-contacting foam gasket 122 such that right-eye sensor 140R and left-eye sensor 140L face the right eye and the left eye of a user, respectively. In other words, right-eye sensor 140R and left-eye sensor 140L are positioned to detect eye movements of the right eye and left eye, respectively. The separate right-eye sensor 140R and left-eye sensor 140L allow for independent tracking of position, movement and line-of-sight directions of eyes of a user using sleep mask device 100. Right-eye sensor 140R and left-eye sensor 140L are routed on separate branches/lines/wires of the flexible interconnect 196 toward the electronic substrate 170 and electronic components thereon, which will be described in further detail with reference to FIG. 6, enabling independent tracking of left and right eyes.

As shown in FIG. 5, on substrate 141, one or more emitters 142 are arranged on and supported by substrate 141. In addition, one or more photodetectors 144 are arranged on and supported by substrate 141. As shown in FIG. 5, the one or more emitters 142 are arranged as an array on substrate 141. Similarly, the one or more photodetectors 144 are arranged as an array on substrate 141. The one or more emitters 142 (which are shown in FIG. 5 placed on substrate 141 within the dot-dash line and outside the dashed line) and the one or more photodetectors 144 (which are shown in FIG. 5 placed on substrate 141 inside the dashed line shown in FIG. 5) which are both arranged on the substrate 141 at positions substantially facing an eye of a user who is wearing sleep mask device 100, when in use. It is to be understood that the emitters 142 and photodetectors 144, are supported and connected to substrate 141 in such a way that facilitates electronic communication between one or more of emitters 142, photodetectors 144, flexible interconnect 196, electronic substrate 170, processor, computer-readable memory, and a power source such as a rechargeable battery or a wired power source.

The one or more emitters 142 include emitters 142A, 142B, 142C, 142D. The emitters 142 are configured to generate, emit, or otherwise produce electromagnetic radiation. Emitter(s) 142 may be configured to emit radiation over any suitable portion of the electromagnetic spectrum, including but not limited to infrared (IR), visible, ultraviolet (UV), or other wavelengths. Emitters 142 may serve any functional purpose such as illumination, signaling, communication, sensing, heating, optical excitation, or other energy transfer operations.

Emitters 142 may be realized using any of a variety of technologies or device structures, including but not limited to: light-emitting diodes (LEDs), including organic (OLED), inorganic, or quantum-dot based LEDs; laser diodes (LDs), including vertical-cavity surface-emitting lasers (VCSELs) or edge-emitting lasers; microLEDs or miniLEDs; photonic integrated circuit (PIC) sources; plasma or discharge lamps; thermal or incandescent emitters; electroluminescent, photoluminescent, or chemiluminescent devices; and any other device capable of producing electromagnetic radiation within a desired wavelength range. Each emitter 142 (emitters 142A, 142B, 142C, 142D) may include one or more active regions, electrodes, contacts, or driver circuits configured to generate electromagnetic radiation when electrically or optically stimulated. Emitters 142 may be driven by a constant or modulated electrical current, voltage, or optical input. In some embodiments, the emitted radiation may be continuous, pulsed, or encoded with modulation patterns for data transmission, sensing, or control. The emission may be directed, collimated, diffused, or shaped through the use of optical structures such as lenses, waveguides, reflectors, or diffusers.

Emitters 142 may be configured to emit radiation at one or more wavelengths, which may be narrowband, broadband, tunable, or otherwise selectable. The peak emission wavelength may range from about 100 nanometers (nm) to about 1 millimeter (mm), depending on the application. Spatially, emitters 142 may be arranged in arrays, matrices, or patterned configurations on a substrate. Such arrays may include single-color or multi-color emitters, arranged to produce a composite emission spectrum or spatial emission pattern.

Emitters 142 may be integrated onto, embedded within, or mounted to a substrate, such as substrate 141, a printed circuit board (PCB), semiconductor wafer, ceramic carrier, or flexible substrate. Electrical interconnection between the emitter and other system components (e.g., drivers, sensors, or controllers) may be provided through conductive traces, vias, or wire bonds. In some embodiments, emitters may be coupled with detectors, sensors, or receivers to form an optoelectronic subsystem, such as a light detection and ranging (LiDAR) unit, optical communication module, display, or sensing system.

As used herein, the term emitter broadly encompasses any device, structure, or system capable of producing electromagnetic radiation in any region of the spectrum, including visible light and non-visible wavelengths. Unless otherwise stated, references to “light” should be understood to include such electromagnetic radiation generally.

The one or more photodetectors 144 include photodetectors 144A, 144B, 144C, 144D, 144E, 144F, 144G, and 144H. The one or more photodetectors 144 are configured to detect electromagnetic radiation emitted by the one or more emitters 142. When the sleep mask device 100 is in use, the electromagnetic radiation emitted by the one or more emitters 142 is incident on an eyelid of a user wearing sleep mask device 100 with their eyes closed. The electromagnetic radiation incident on the eyelid is reflected by the eyelid and then received by the photodetectors 144. Photodetectors 144 may include any suitable light-sensitive element capable of generating an electrical signal in response to incident radiation.

In various embodiments, the photodetectors are configured to detect radiation within one or more spectral bands, such as visible, infrared (IR), near-infrared (NIR), or ultraviolet (UV) wavelengths, depending on the characteristics of the emitter and the desired application. Each of the one or more photodetectors 144 (photodetectors 144A, 144B, 144C, 144D, 144E, 144F, 144G, and 144H) may comprise a photosensitive semiconductor device, such as a photodiode, phototransistor, avalanche photodiode (APD), or photoconductive cell. Furthermore, it should be understood that photodetectors described in this description and illustrated as single elements in the drawings are not necessarily single photo-sensitive elements. Each individual photodetector described herein can be made up of an array or grid of multiple arranged photosensitive elements that each receive electromagnetic radiation and contribute to the sensor output of the photodetector. In some embodiments, the photodetector(s) 144 includes a semiconductor junction (e.g., p-n, p-i-n, or Schottky) formed within a substrate material such as silicon, germanium, indium gallium arsenide (InGaAs), or another compound semiconductor suitable for the intended wavelength range. The photodetector(s) 144 may optionally include optical filters, lenses, or collimators positioned to selectively pass or focus radiation onto the active region. The output of each photodetector may be electrically coupled to a signal conditioning circuit, amplifier, analog-to-digital converter, or processing module configured to interpret the detected signal.

During operation, the one or more emitters 142 can all collectively generate electromagnetic radiation that propagates toward the closed eyelid of a user wearing sleep mask device 100, reflects off the eyelids and is received by photodetectors 144. Not all emitters 142 need to emit electromagnetic radiation at the same time. Each individual emitter 142A, 142B, 142C, 142D can be controlled to emit electromagnetic radiation alone individually, or a subset of the emitters 142, in any combination, can be controlled to emit electromagnetic radiation. The emitted electromagnetic radiation from one, all, or a subset of emitters 142 reflects off of the eyelids of the user and is then received by one or more photodetectors 144. During operation, the one or more photodetectors 144 can all collectively receive and produce sensor signals based on the received electromagnetic radiation that propagates toward the closed eyelid of a user wearing sleep mask device 100 and reflects off the eyelids. Not all photodetectors 144 need to be actuated or activated to receive electromagnetic radiation at the same time. Each individual photodetectors 144A, 144B, 144C, 144D, 144E, 144F, 144G, and 144H can be controlled to receive electromagnetic radiation alone individually, or a subset of the photodetectors 144, in any combination, can be controlled to be activated to receive electromagnetic radiation. The received electromagnetic radiation from one, all, or a subset of photodetectors 144 can then be converted to sensor signals and output to a processor.

One or more photodetectors 144 absorb a portion of the incident radiation and convert it into a measurable electrical response, such as a photocurrent or voltage proportional to the radiation intensity. In some embodiments, the photodetector response may be used to determine one or more parameters associated with the emitters 142 or the environment, such as emission intensity, modulation frequency, spatial position, or temporal variations in radiation output. The photodetectors 144 may be arranged in a linear, two-dimensional, or three-dimensional array to provide spatial resolution of the detected radiation pattern. In certain embodiments, multiple photodetectors are oriented at different angles or positions relative to the emitters 142 to detect radiation from various directions or to enable triangulation, imaging, or pattern recognition. Based on the electromagnetic radiation received by photodetectors 144 is output by photodetectors 144 as an electrical signal to a processor.

The photodetectors 144 and emitters 142 may be mounted on a common substrate, within a shared housing, or on opposing sides of a medium or optical path. Electrical interconnections between the photodetectors and associated circuitry may be implemented using conductive traces, flex circuits, or wire bonds.

FIG. 6 is a functional block diagram of an example head-mounted sleep mask device, in accordance with one or more embodiments of the present disclosure. As shown in FIG. 6, sleep mask device 100 may include a computing device 102 operatively coupled to one or more peripheral components of the sleep mask device 100. In some embodiments, computing device 102 comprises one or more processors and one or more non-transitory computer-readable memories storing instructions that, when executed by the one or more processors, cause computing device 102 to perform operations described herein, including monitoring user eye activity, determining eye position and/or eye movement of a closed eye, and controlling one or more output modalities to facilitate sleep, relaxation, or other user experiences.

Sleep mask device 100 may further include eye tracker sensor 140 configured to detect signals associated with a user's eye position, eye movement, eyelid contour, and/or a spatial intensity pattern derived from electromagnetic radiation reflected from the eye region. In the illustrated embodiment, eye tracker sensor 140 includes left eye tracker sensor 140L and right eye tracker sensor 140R. Left eye tracker sensor 140L and Right eye tracker sensor 140R may be arranged to correspond to the user's left and right eyes, respectively, and may generate sensor data provided to computing device 102 for processing. In some embodiments, computing device 102 determines an eye position (or a change in eye position over time) based on the sensor data and maps the determined eye position to one or more operational commands that trigger a function of sleep mask device 100.

Sleep mask device 100 may include one or more audio output devices, such as speakers 150. In the illustrated embodiment, speakers 150 include a left speaker 150L and a right speaker 150R. Computing device 102 may drive speakers 150 to output audio, such as sleep-inducing sounds, guided relaxation content, binaural beats, masking noise, alarms, prompts, or other audio content. Left speaker 150L and Right speaker 150R may be independently controlled to provide stereo playback, spatialized audio, balance adjustment, and/or individualized output to each ear.

Sleep mask device 100 may further include microphone 160 operatively coupled to computing device 102. Microphone 160 may be configured to capture one or more acoustic signals, including user speech, breathing, snoring, ambient noise, or other sound. In some embodiments, microphone signals are used by computing device 102 for noise monitoring, adaptive audio control, voice command detection, sleep-state estimation, user interaction, or calibration and diagnostic procedures.

Sleep mask device 100 may be powered by an internal power source such as a battery 176. Battery 176 may be rechargeable and electrically coupled to computing device 102 and/or other components of the sleep mask device 100 to provide operating power. The sleep mask device 100 may further include a power input interface 178 configured to receive power from an external power source 600, for example to charge battery 176 and/or to power sleep mask device 100 during operation. Power input interface 178 may include, by way of example, a wired connector, charging port, magnetic connector, or inductive charging interface. In some embodiments, computing device 102 manages charging operations, power distribution, battery monitoring, and/or transitions between external power and battery power.

Interconnections illustrated in FIG. 7 between computing device 102 and eye tracker sensor 140, speakers 150, microphone 160, battery 176, and power input interface 178 may represent any suitable electrical, data, and/or control coupling, including one or more buses, wired connections, flexible circuits, or other interconnect structures. Although the components are depicted as discrete functional blocks, one or more components may be integrated with other components, combined, or distributed across multiple modules. Additionally, the particular set of components shown in FIG. 7 is exemplary, and additional components (e.g., wireless transceivers, haptic actuators, additional sensors, light emitters, or user input devices) may be included in other embodiments.

Referring now to FIG. 7, an example implementation of computing device 102 of sleep mask device 100 is shown in greater detail. Computing device 102 may comprise one or more processors 712, one or more computer-readable memory devices 720, one or more input/output (I/O) devices 714, and one or more networking devices 716, which may be communicatively coupled via one or more communication paths or buses 718.

Processor(s) 712 may include any suitable processing circuitry, such as one or more microprocessors, microcontrollers, digital signal processors (DSPs), application-specific integrated circuits (ASICs), field-programmable gate arrays (FPGAs), system-on-chip (SoC) devices, or combinations thereof. Processor(s) 712 may be configured to execute instructions to perform operations described herein, including processing eye tracking data, determining eye position or eye movement of a closed eye, mapping detected eye positions to operational commands that trigger operations or functions of the sleep mask device 100, and controlling one or more output devices of sleep mask device 100.

Memory device(s) 720 may include one or more non-transitory computer-readable storage media, such as volatile memory (e.g., RAM), non-volatile memory (e.g., flash memory, EEPROM), or combinations thereof. Memory device(s) 720 may store executable instructions, firmware, software, data, or combinations thereof. In the illustrated embodiment, memory device(s) 720 include a data store 722 and one or more modules 724.

Data store 722 may be configured to store data used or generated by computing device 102, including but not limited to eye tracking sensor data, spatial intensity patterns, eye position determinations, calibration parameters, user preferences, audio content, control mappings, usage history, and diagnostic or state information. In some embodiments, data store 722 may store multiple spatial intensity patterns captured over time and associated eye position information to enable tracking of eye movement or changes in eye position over time.

Modules 724 may include software, firmware, or logic modules that, when executed by processor(s) 712, cause computing device 102 to perform one or more functions of the sleep mask device 100. By way of non-limiting example, modules 724 may include one or more of: an eye tracking module for eye position detection and direction detection of eye movement, a signal processing module, a spatial intensity analysis module, an eye position determination module, a command mapping module, an audio control module, an audio generation module, an interactive narrative engine, a user task generation module, a spatial audio module, an external device control module, an adaptive sleep-tapering module, a power management module, a user interaction module, and/or a sleep program execution module. Although illustrated as discrete modules, the functionality of modules 724 may be combined, distributed, or otherwise arranged across one or more software components.

I/O devices 714 may include one or more interfaces configured to facilitate communication between computing device 102 and external or peripheral components. In the illustrated embodiment, I/O devices 714 may interface with eye tracker sensor 140, speakers 150, microphone 160, and battery 176. I/O devices 714 may include analog-to-digital converters, digital-to-analog converters, sensor interfaces, audio drivers, power management circuitry, or other suitable interface components.

Networking devices 716 may optionally include one or more wired or wireless communication interfaces, such as Bluetooth®, Wi-Fi®, near-field communication (NFC), or other communication technologies. In some embodiments, networking devices 716 enable communication between sleep mask device 100 and an external device, such as a smartphone, tablet, or computing system, for purposes including configuration, content delivery, data synchronization, firmware updates, or remote control. In other embodiments, networking devices 716 may be omitted.

Bus 718 may represent one or more data buses, control buses, power buses, or combinations thereof that facilitate communication among processor(s) 712, memory device(s) 720, I/O devices 714, and networking devices 716. Although illustrated as a single bus, multiple buses or interconnect structures may be used.

As shown in FIG. 7, computing device 102 may be operatively coupled to eye tracker sensor 140, speakers 150, microphone 160, and battery 176 of sleep mask device 100. Computing device 102 may receive sensor data from eye tracker sensor 140, process the sensor data to determine eye position or eye movement, and generate control signals to drive speakers 150 or other output devices. Computing device 102 may further receive acoustic signals from microphone 160 and manage power delivery, charging, and battery state via battery 176.

Referring back to FIG. 4 in connection with FIGS. 6 and 7, the various electronic components mentioned herein can be in electronic communication with electronic substrate 170 shown in FIG. 4 and configured to operate, via signals based on the instructions stored in memory devices 720, executed by processor 712, and relayed via electronic pathways on the electronic substrate 170 or wirelessly via any suitable wireless communication interface.

The architecture illustrated in FIG. 7 is provided by way of example, and variations are contemplated. One or more components may be integrated, omitted, duplicated, or rearranged without departing from the scope of the present disclosure. Additionally, certain functionality described as being performed by computing device 102 may, in other embodiments, be performed by dedicated hardware, distributed processing elements, or remote computing resources.

### Eye Position and Movement Detection

FIG. 8A-8C are depictions of closed-eye reflectance sensing, showing emitter illumination of the eyelid, photodetector reception of the emitted electromagnetic radiation, and example sensor outputs in which spatial intensity varies with left, center, and right closed-eye positions. In other words, FIGS. 8A-8C illustrates examples of detecting an eye position of a closed eye using eye tracker sensor 140 incorporated into sleep mask device 100. As described elsewhere in this description, and as shown in FIG. 5, eye tracker sensor 140 (e.g., right-eye sensor 140R and left-eye sensor 140L) may include one or more emitters 142 configured to emit electromagnetic radiation and one or more photodetectors 144 configured to receive electromagnetic radiation reflected from, or transmitted through, an eyelid of the user when the mask portion is placed over the user's eyes. FIGS. 8A-8C show an example of at least one emitter 142B and at least one photodetector 144D, as also shown in FIG. 5, that can be used to track position, movement, and direction of a closed eye of a user. As depicted in FIGS. 8A-8C, the eyelid covering the eye forms a contour influenced by the underlying eyeball. Because an eyeball beneath a closed eyelid presents a generally dome-shaped surface, portions of the eyelid closer to the one or more emitters 142 and the one or more photodetectors 144 reflect or transmit electromagnetic radiation with greater detected intensity than portions that are farther away. Accordingly, the spatial distribution of detected electromagnetic radiation correlates with the contour of the eyelid, which in turn correlates with the underlying position of the eye. The eye position detection described herein is performed by physical sensors and electronic processing circuitry and does not rely on human observation or mental interpretation.

FIG. 8A illustrates, from left to right, closed eyes in a right-gazing position, a center-gazing position, and a left-gazing position relative to the user's point of view. With the eyes closed, it is not apparent where the cornea of the eye is positioned underneath the eyelid. A center line C indicates an approximate center of the closed eyes. Because the gazing direction or eye position is not apparent due to the closed eyes, the eye tracker sensor including emitter 142B and photodetector 144D are used to track the position of the closed eyes. With the user's eyes closed, emitter 142B emits electromagnetic radiation on the eyelid of the user's eyes, the electromagnetic radiation reflects off the eyelid and is received by the photodetector 144D. As the eyeball moves beneath the eyelid, the eyelid's external contour and tension change slightly, which produces spatial and intensity variations in reflected light received at the detectors 144.

FIG. 8B shows example pupil/cornea or eyeball positions of a closed eye, such as left-gazing, center-gazing, and right-gazing as labeled in FIG. 8B. Cornea position 801A shown in FIG. 8B illustrates the position of the cornea of the eye of the user under the eyelid when the user is gazing rightward. From the user's point of view, cornea position 801A is a right-gazing eye position where the eye is to the right of center line C, and to the left of center line C from the point of view of a viewer of FIG. 8B. Cornea position 801B shown in FIG. 8B illustrates the position of the cornea of the eye of the user under the eyelid when the user is gazing in a center direction. From the user's point of view, cornea position 801B is a center-gazing eye position where the eye is substantially centered on the center line C to both the user of sleep mask device 100 and a viewer of FIG. 8B. Cornea position 801C shown in FIG. 8B illustrates the position of the cornea of the eye of the user under the eyelid when the user is gazing leftward. From the user's point of view, cornea position 801C is a left-gazing eye position where the eye is to the left of center line C, and to the right of center line C from the point of view of a viewer of FIG. 8B.

FIG. 8C shows a plurality of spatial intensity patterns based on sensor signals from photodetector 144D, which sensor signals are based on received electromagnetic radiation that was emitted from emitter 142B and reflected off the eyelid of the user to photodetector 144D. The spatial intensity patterns shown correspond to the eye pupil or cornea positions of a closed eye of right-gazing, center-gazing, and left-gazing eye positions shown in FIGS. 8A and 8B and located above the spatial intensity patterns of FIG. 8C.

Intensity scale 803 shows light intensity levels detected by photodetector 144D and illustrated in spatial intensity patterns 802A, 802B, and 802C. Each square on intensity scale 803 is associated with a particular intensity of electromagnetic radiation detected by photodetector 144D. In the example of FIGS. 8A-8C, the intensity level of intensity scale 803 indicated by a white square indicates intensity levels that correlate with a position of a cornea of an eye of the user. For example, depending on environment and circumstances surrounding the user and sleep mask device 100, higher intensity values may be associated with the position of the cornea of the user. In such case, the white squares would indicate the position of the cornea and the white squares would be higher intensity squares compared to the darker squares. Again, depending on environment, it is possible that lower intensity squares may be indicative of cornea position, in which case the white squares would be lower intensity squares compared to the darker squares.

Photodetector 144D and emitter 142B are positioned obliquely with respect to the eye of the user as shown in FIG. 8B and as further illustrated by the positions emitter 142B and photodetector 144D on substrate 141 as shown in FIG. 5. Such an arrangement leads to the spatial intensity patterns shown in FIG. 8C. Spatial intensity pattern 802A corresponds to a spatial intensity pattern generated when the cornea of the user is in a right-gazing cornea position 801A. As shown in spatial intensity pattern 802A, the intensity portion indicated by white squares extends to a leftward side of spatial intensity pattern 802A, as viewed by a viewer of FIG. 8C, which corresponds to a right-gazing position of the eye from the point of view of a user of the sleep mask device 100. Spatial intensity pattern 802B corresponds to a spatial intensity pattern generated when the cornea of the user is in a center-gazing cornea position 801B. As shown in spatial intensity pattern 802B, the intensity portion indicated by white squares extends to a central portion of spatial intensity pattern 802B, as viewed by both a viewer of FIG. 8C and from the point of view of a user of the sleep mask device 100. Spatial intensity pattern 802C corresponds to a spatial intensity pattern generated when the cornea of the user is in a left-gazing position. As shown in spatial intensity pattern 802C, the intensity portion indicated by white squares is located in a rightward side of spatial intensity pattern 802C, as viewed by a viewer of FIG. 8C, which corresponds to a left-gazing cornea position 801C of the eye from the point of view of a user of the sleep mask device 100.

The spatial intensity patterns (e.g., 802A, 802B, and 802C) can be produced in the following way. The electromagnetic radiation reflecting off the eyelid and received by the photodetector 144D is converted to a sensor signal, or, in other words, an eye position signal,

by the photodetector 144D that can be output to computing device 102 and processor 712 for analysis. The eye position signal can be output to the processor 712 as raw sensor signals that the processor then analyzes to create a spatial intensity pattern (e.g., 802A, 802B, and 802C). The eye position signal output by photodetector 144D is indicative of an eye position of one or more eyes of the user wearing sleep mask device 100. The processor 712 receives the eye position signal and can analyze the eye position signal to determine the eye position of the user of sleep mask device 100.

The eye position signal is indicative of the eye position of the user because the pattern and/or reflectance of the reflecting electromagnetic radiation off the eyelid is caused by contour variations in the eyelid caused by the dome shape of the cornea beneath the eyelid, which in turn causes different distances between the different areas of the eye and the photodetectors 144 receiving the electromagnetic radiation reflecting off the eye. The photodetectors 144 convert the the reflecting electromagnetic radiation to the eye position signal for analysis by the processor 712 and determination of the eye position of the user by the processor. For clarity in defining the different terms, it is to be understood that “eye position signal” refers to raw or conditioned photodetector output or sensor data from the photodetectors 144. The term “spatial intensity pattern” is a processor 712 generated representation of the eye position signal. The term “eye position profile” is a stored calibration reference or preset profile associated with a particular position of an eye and is stored for comparison to spatial intensity patterns generated by the processor 712 based on the eye position signal from the eye-tracker sensor 140.

As will be described in further detail below, spatial intensity pattern 802A can be determined by analysis performed by processor 712, based on instructions stored in computer-readable storage memory, to be associated with the right-gazing eye position. As such, a position of a user's eye can be detected based on a processor 712 analyzing spatial intensity pattern 802A and determining the position of the eye to be a right-gazing position based on the association of spatial intensity pattern 802A with the right-gazing position. Similarly, spatial intensity patterns 802B and 802C can be determined by analysis performed by processor 712, based on instructions stored in computer-readable storage memory, to be associated with a center-gazing position or left-gazing position, and positions of a user's eye can be detected based on processor 712 analyzing spatial intensity patterns 802B and 802C and determining the position of the eye to be a center-gazing position or left-gazing position based on the association of spatial intensity pattern 802B and 802C with the center-gazing position and left-gazing position as determined by processor 712.

In the example of FIGS. 8A-8C, the cornea position and, therefore, position of the eye of the user, is detected based on intensity of electromagnetic radiation emitted by an emitter 142, reflected by the eyelid, and received by the photodetector 144. However, it is to be appreciated that intensity is only one example of a characteristic that can be used to detect eye shape and cornea position beneath an eyelid. The spatial intensity pattern of electromagnetic radiation reflected from a closed eyelid arises from geometric and anatomical contour variations of the eyelid that are induced by underlying ocular structures. In particular, the generally dome-shaped geometry of the cornea produces a non-uniform surface profile in the overlying eyelid tissue. Any sensor characteristic or detection functionality that can indicate eye shape and a position of a cornea under a closed eye can be used to detect eye position of a closed eye.

In the example of FIGS. 8A-8C, the non-uniform surface profile of an eye under an eyelid causes different regions of the eyelid to be positioned at different distances and angular orientations relative to one or more photodetectors 144 of the sleep mask device 100. As one example of detecting eye position, electromagnetic radiation emitted toward the eyelid and reflected therefrom exhibits spatially varying intensity levels when received by the photodetectors 144. In at least one example, regions of the eyelid that are positioned closer to the photodetectors 144, or that present a surface normal more directly aligned with the optical axis of a given photodetector, may produce higher detected intensity values, while regions positioned farther away or oriented at steeper angles may produce lower detected intensity values. Accordingly, the received electromagnetic radiation forms a spatial intensity pattern across the photodetector array that corresponds to the underlying three-dimensional shape of the eye beneath the eyelid.

In some embodiments, movement of the eyeball beneath the eyelid—such as rotation of the cornea during eye motion—causes a corresponding shift or deformation of the eyelid contour. This, in turn, produces a temporal change in the spatial intensity pattern based on the eye position signal of the photodetectors 144. By analyzing one or more spatial intensity patterns captured over time, the system may determine an eye position, eye movement direction, or eye movement trajectory of the user, even when the eye remains closed.

Additionally or alternatively, the spatial intensity pattern may be influenced by factors including eyelid thickness, tissue elasticity, reflectivity of the eyelid surface, wavelength of the emitted electromagnetic radiation, and the spatial arrangement of the photodetectors 144. Nonetheless, the dome-shaped corneal structure remains a primary contributor to the non-uniform eyelid contour that gives rise to the detectable spatial intensity variations used for eye position sensing.

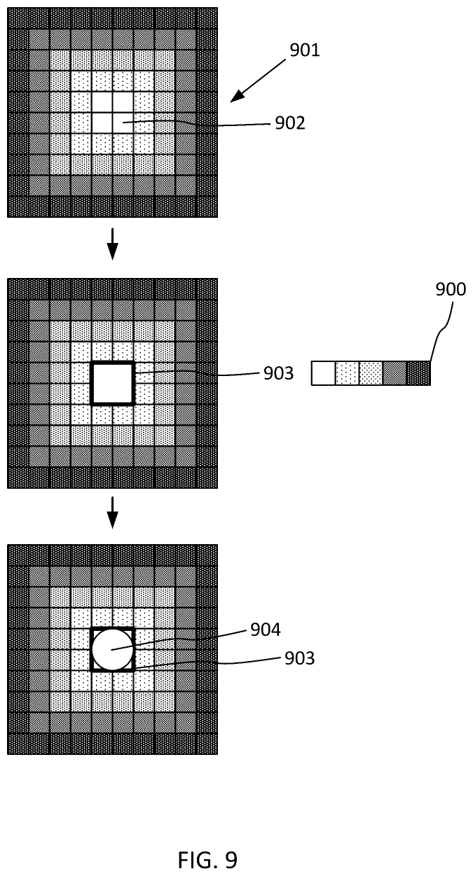

FIG. 9 is an illustration of detecting closed eye position of the eyes of a user based on sensor output of an eye position signal by a photodetector based on received electromagnetic radiation and eye position signal analysis by a processor. FIG. 9 shows an exemplary spatial intensity pattern 901. Spatial intensity pattern 901 can be generated by any of the methods described herein. For example, emitters 142 can emit electromagnetic radiation towards a closed eyelid of a user and the electromagnetic radiation can be reflected off the eyelid and received by photodetectors 144. Photodetectors 144 can output an eye position signal to computing device 102 and processor 712. Processor 712 can then analyze the eye position signal to produce spatial intensity pattern 901. Intensity scale 900 indicates the intensity levels of electromagnetic radiation represented in the individual squares of the grid shown as spatial intensity pattern 901. For purposes of this example, white squares in the intensity scale 900 are higher intensity values and shaded squares are lower intensity values depending on the level of shading. Darker squares are lower intensity than lighter squares.

As shown in spatial intensity pattern 901, the central 2×2 section 902 of the grid is of the highest intensity when compared to the rest of the spatial intensity pattern 901. The processor 712, when analyzing spatial intensity pattern 901, identifies the central 2×2 section 902 as a higher intensity section 903 within the spatial intensity pattern 901 and determines that the higher intensity section is a likely location of the cornea of the user's eye. The processor identifies this section as an eye location 904 of the user's eye within the spatial intensity pattern 901.

There are several methods the processor 712 can then use to determine the eye position of the user. Firstly, in some embodiments, determining the eye position of the eye beneath the closed eyelid includes identifying a location (e.g., higher intensity section 903) within the detected spatial intensity pattern 901 that corresponds to a highest detected intensity of the reflected electromagnetic radiation in the spatial intensity pattern 901. The processor 712 can then utilize a coordinate system of the spatial intensity pattern 901 to determine which coordinates within the spatial intensity pattern 901 exhibit the higher intensity section 903. The coordinates of the spatial intensity pattern 901 can be associated in memory devices 720 with specific locations, quadrants, section or otherwise, such as, for example, up, down, left, right, center, diagonal upward right, diagonal upward left, diagonal downward right, diagonal downward left. Accordingly, when the processor 712 determines that the higher intensity section 903 is in coordinates associated with a center position, the processor 712 determines that the eye of the user is in a center gazing position and then controls the sleep mask device 100 in accordance with any rules, commands, or operations associated with the eye being in the center gazing position.

As another example method the processor 712 can use to determine the eye position of the user, memory devices 720 can store eye position profiles, each associated with a specific eye position of the user. The processor 712 can compare a spatial intensity pattern (e.g. spatial intensity pattern 901) to the stored eye position profiles. The processor 712 can match the spatial intensity pattern to an eye position profile and determine the eye position of the user based on the match between spatial intensity pattern and eye position profile. In the case there are no exact matches, the processor 712 can find a best matching eye position profile for a given spatial intensity pattern. For example, the processor 712 can calculate a best match between eye position profile for a given spatial intensity pattern by determining if the intensity values and locations match between an eye position profile and a given spatial intensity pattern within a certain predefined margin of error.

In some embodiments, determining whether two spatial intensity patterns are substantially similar is performed by a processor based on one or more comparison criteria, including but not limited to correlation values, distance metrics, pattern overlap, relative intensity distributions, normalized intensity differences, weighted region comparisons, threshold comparisons, confidence scores, or combinations thereof. In some embodiments, two spatial intensity patterns are considered substantially similar when a similarity metric computed by the processor satisfies a predetermined threshold or range associated with a corresponding eye position profile. In some embodiments, substantial similarity may be determined based on comparison of multiple spatial intensity patterns collected over time, including averaged, filtered, or time-weighted representations of such patterns. Determinations of substantial similarity as described herein are performed by electronic processing circuitry and are not based on human observation or subjective judgment.

In another exemplary method, the highest-intensity point of the spatial intensity pattern, as determined by the processor 712, may be used as an indicator of the portion of the eyelid that is closest to the one or more emitters and one or more photodetectors, and therefore may correspond to the direction in which the eye is oriented beneath the eyelid.

In other embodiments, the processor may determine the eye position based on a region rather than a single point. For example, the processor may identify a plurality of areas within the spatial intensity pattern that exhibit the highest or near-highest intensity values. An average, centroid, weighted centroid, or other aggregate position of these areas of elevated intensity may then be computed. The resulting aggregate position may be used as the detected eye position. Such approaches may improve robustness in cases where the reflected intensity peak is broader, less sharply defined, or affected by noise, eyelid characteristics, or individual anatomical differences.

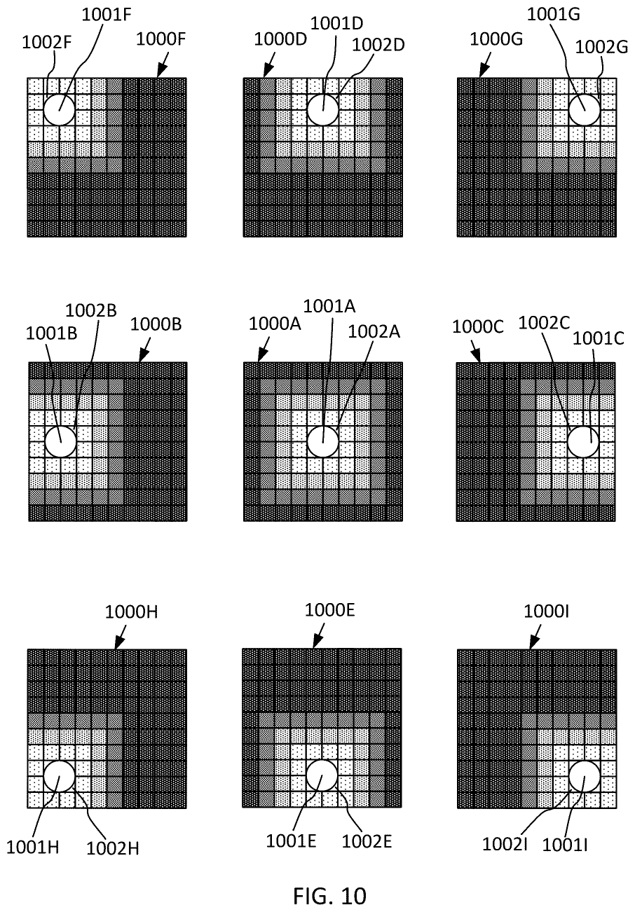

FIG. 10 illustrates several exemplary sensor outputs, spatial intensity patterns, and eye position profiles that are respectively detected by a photodetector, analyzed by a processor, and associated with closed-eye positions of eyes of a user of an exemplary head-mounted electronic sleep mask device 100. It is to be understood that each of the illustrations in FIG. 10 can be considered either a sensor output from photodetector(s) 144, or a spatial intensity pattern that is analyzed by the processor 712, or an eye position profile that may be pre-saved in computer-readable memory for comparison with sensor output and/or spatial intensity patterns. For example, in the center of FIG. 10 is an intensity pattern 1000A. Intensity pattern 1000A can represent a sensor output directly from photodetector(s) 144. Alternatively, intensity pattern 1000A can represent a spatial intensity pattern analyzed by processor 712 based on an eye position signal output from photodetector(s) 144. Alternatively, intensity pattern 1000A can represent an eye position profile that is pre-saved in memory devices 720 for later comparison with gathered eye position signals output from photodetector(s) 144 and/or spatial intensity patterns generated and/or analyzed by the processor 712 to detect the eye position of the user of sleep mask device 100 based on the comparison between the stored eye position profile and eye position signal/spatial intensity pattern. The other intensity patterns 1000A, 1000B, 1000C, 1000D, 1000E, 1000F, 1000G, 1000H, and 1000I in FIG. 10 can similarly be considered either sensor outputs, spatial intensity patterns, and/or eye position profiles.

Each of intensity patterns 1000A, 1000B, 1000C, 1000D, 1000E, 1000F, 1000G, 1000H, and 1000I shown in FIG. 10 include a representation of an eye location (i.e., 1001A, 1001B, 1001C, 1001D, 1001E, 1001F, 1001G, 1001H, and 1001I, as well as a representation of a higher intensity section (i.e., 1002A, 1002B, 1002C, 1002D, 1002E, 1002F, 1002G, 1002H, and 1002I). Each of intensity patterns 1000A, 1000B, 1000C, 1000D, 1000E, 1000F, 1000G, 1000H, and 1000I is associated with a specific eye location and therefore indicates a specific eye position of the user using sleep mask device 100.

Intensity pattern 1000A illustrates an eye location 1001A in a center position and is associated with detecting an eye position of the user of the sleep mask device 100 being at a center-gazing position. Intensity pattern 1000B illustrates an eye location 1001B in a leftward position and is associated with detecting an eye position of the user of the sleep mask device 100 being at a left-gazing position. Intensity pattern 1000C illustrates an eye location 1001C in a rightward position and is associated with detecting an eye position of the user of the sleep mask device 100 being at a right-gazing position. Intensity pattern 1000D illustrates an eye location 1001D in an upward position and is associated with detecting an eye position of the user of the sleep mask device 100 being at an upward-gazing position. Intensity pattern 1000E illustrates an eye location 1001E in a downward position and is associated with detecting an eye position of the user of the sleep mask device 100 being at a downward-gazing position. Intensity pattern 1000F illustrates an eye location 1001F in an upward-leftward position, and is associated with detecting an eye position of the user of the sleep mask device 100 being at a upward-leftward gazing position. Intensity pattern 1000G illustrates an eye location 1001G in an upward-rightward position and is associated with detecting an eye position of the user of the sleep mask device 100 being at an upward-rightward gazing position. Intensity pattern 1000H illustrates an eye location 1001H in a downward-leftward position and is associated with detecting an eye position of the user of the sleep mask device 100 being at a downward-leftward gazing position. Intensity pattern 1000I illustrates an eye location 1001I in a downward-rightward position and is associated with detecting an eye position of the user of the sleep mask device 100 being at a downward-rightward gazing position.

As illustrated in each of the intensity patterns of FIG. 10, lighter or white areas represent higher detected intensities, and increasingly dense shaded represents lower detected intensities. When the user's eye is positioned toward the right, (or in other words, a right-gazing position), a higher-intensity region appears on the right side of the intensity pattern; when the eye is positioned toward the left (or in other words, a left-gazing position), the higher-intensity region appears on the left; and when the eye is centered, (or in other words a center-gazing position), the highest-intensity region appears near the center. Upward or downward gazing eye positions similarly cause upward or downward shifts in the location of the highest-intensity region. Similarly, diagonal-upward-gazing or diagonal-downward-gazing eye positions similarly cause diagonal upward or diagonal downward shifts in the location of the highest-intensity region. Thus, the spatial and intensity variations in detected electromagnetic radiation correspond to changes in eyelid contour caused by the underlying eye at different positions. Any location of the higher intensity region is within the scope of this disclosure for determining the position of the eyeball beneath the eyelid. In other words, any eye-gazing position located between the up, down, left, right, center, diagonal upward, and diagonal downward positions are intended to be detectable based on the location of the higher intensity region in the spatial intensity pattern(s) detected by the photodetector(s), utilizing the processor, memory, and executable instructions stored therein.

The processor 712 in electronic communication with the eye tracker sensor is configured to analyze the eye position signals generated by the one or more photodetectors 144. Using instructions stored in a non-transitory computer-readable medium of memory device 720, the processor 712 may determine one or more positions of the user's eyes based on these spatial and intensity variations shown in FIG. 10.

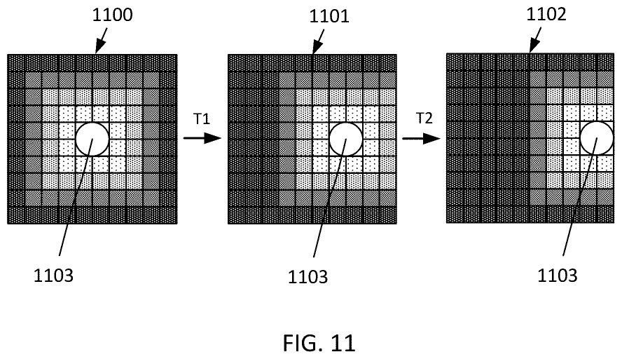

FIG. 11 illustrates gathering of multiple sequential spatial intensity patterns over a period of time. For example, as the emitter(s) 142 continuously emit electromagnetic radiation on to the eyelid, the photodetector(s) 144 continuously receive the reflected electromagnetic radiation and produce/output eye position signals at a preset sampling rate. During this process, a first eye position signal is used by the processor 712 to generate a first spatial intensity pattern 1100. After a first time period T1, emission of electromagnetic radiation and outputting eye position signals continues. After T1, a second eye position signal is used to generate a second spatial intensity pattern 1101. After a second time period T2 a third eye position signal is used to generate a third spatial intensity pattern 1102. As shown in FIG. 11, each of the spatial intensity patterns 1100, 1101, and 1102 include an eye location represented therein 1103 that translates over time from a center position to a more rightward position after T1 and to an even more rightward position after time T2. The sleep mask device 100 can operate such that multiple sequential spatial intensity patterns 1100, 1101, and 1102 are generated/analyzed by the processor and stored in memory devices 720. With multiple spatial intensity patterns 1100, 1101, and 1102 collected over a known period of time and stored for analysis, it becomes possible to determine eye movement direction (i.e., comparing starting position to ending position of the eye), eye movement velocity (i.e., distance traveled over a period of time), eye acceleration (i.e., change of speed over time).

This temporal analysis may enable detection of voluntary or involuntary eye motions, including saccades, fixations, blinks, micro-movements, or patterns associated with REM or non-REM sleep states. In some embodiments, motion trajectories determined from the time series of spatial intensity patterns (e.g., those shown in FIG. 11) may be used by the processor to modify audio output, navigate audio scenes, control interactive content, or detect user intent based on the movement of the eye beneath the eyelid, as will be described in further detail below.

FIG. 12 illustrates a simplified functional block diagram that shows how sleep mask device 100 can operate and communicate with external devices. Referring now to FIG. 12, sleep mask device 100 is shown in communication with one or more external devices via the networking devices 816 of the computing device 102. In the illustrated embodiment, the networking devices 816 enable both wired and wireless communication between the sleep mask device 100 and one or more external electronic devices. The networking devices 816 may include one or more communication interfaces configured to support wireless communication, wired communication, or a combination thereof. By way of non-limiting example, the networking devices 816 may support Bluetooth®, Bluetooth Low Energy (BLE), Wi-Fi®, near-field communication (NFC), ultra-wideband (UWB), cellular communication, proprietary wireless protocols, wired serial communication, or combinations thereof.

As illustrated in FIG. 12, the networking devices 816 may include a wired communication interface 816A and a wireless communication interface 816B. The wired communication interface 816A may enable communication with external devices via a physical connection, such as a cable, docking interface, or shared charging and data interface. The wireless communication interface 816B may enable radio-frequency communication with one or more external devices without a physical connection. The sleep mask device 100 may communicate with a personal electronic device 1200, such as a smartphone, tablet, laptop, or other user computing device. In some embodiments, the personal electronic device 1200 executes a companion application that communicates with the sleep mask device 100 to configure device settings, initiate or modify sleep programs, upload or download audio content, receive sleep or eye-tracking data, provide firmware updates, log sessions, or enable user interaction and control.

The sleep mask device 100 may further communicate with an activity tracker 1202, which may include a wearable device configured to monitor physiological or activity-related parameters of the user, such as motion, heart rate, respiration, sleep stage, or other biometric data. In some embodiments, data received from the activity tracker 1202 may be used by the computing device 102 to adjust operation of the sleep mask device 100, such as modifying audio output, sleep program timing, eye-based command interpretation, or power management behavior.

The sleep mask device 100 may also communicate with an external speaker 1204, which may be separate from the speakers 150 integrated into the sleep mask device 100. In some embodiments, the computing device 102 transmits audio signals to the external speaker 1204 via the networking devices 816, thereby enabling audio output to be delivered through one or more remote or room-based speakers. This may allow, for example, sleep-related audio content to be played at a desired location or volume separate from the sleep mask device 100.

Communication between the sleep mask device 100 and the external devices 1200, 1202, and 1204 may be bidirectional or unidirectional, continuous or intermittent, and may occur in real time or near-real time. The computing device 102 may manage pairing, authentication, synchronization, data formatting, and communication timing with the external devices via the networking devices 816 and the I/O devices 814. Although FIG. 12 illustrates communication with a personal electronic device 1200, an activity tracker 1202, and an external speaker 1204, this configuration is provided by way of example only. In other embodiments, the sleep mask device 100 may communicate with additional or alternative external devices, such as cloud-based services, home automation systems, alarm systems, lighting systems, or other network-connected devices. Furthermore, one or more of the external devices shown may be omitted, combined, or replaced without departing from the scope of the present disclosure.

### User Calibration for Detecting Eye Positions

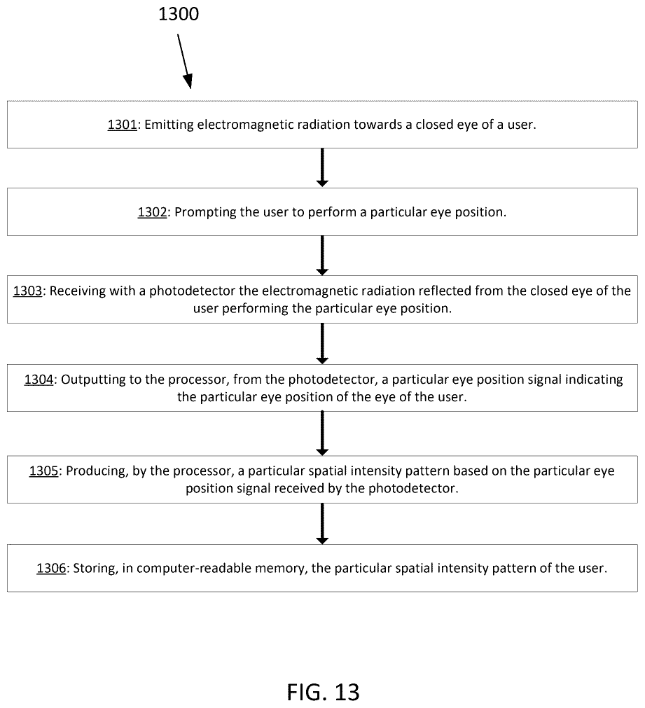

The system can perform a brief per-user calibration process to associate observed reflection of electromagnetic radiation patterns off an eye of a user with specified eye positions of a closed eye. Referring now to FIG. 13, method 1300 is shown for calibrating the sleep mask device to a specific user. The method 1300 may be performed by the sleep mask device 100, and in particular by the computing device 102 and processor 712, using one or more emitters 142 and photodetectors 144 positioned to interact with a closed eye of the user.

At step 1301, the method includes emitting electromagnetic radiation toward a closed eye of a user. In some embodiments, one or more emitters 142 emit electromagnetic radiation toward a closed eyelid of the user while the sleep mask device 100 is worn. The electromagnetic radiation may include infrared radiation, near-infrared radiation, or other wavelengths suitable for transmission through or reflection from the eyelid. The electromagnetic radiation may illuminate at least a portion of the eyelid and underlying ocular structures, including the cornea.

At step 1302, the method includes prompting the user to perform a particular eye position. The prompt may be provided via one or more output modalities of the sleep mask device 100, including audio output through speakers 150, haptic feedback, or other sensory cues. The particular eye position may correspond to a predefined calibration position, such as looking left, right, up, down, center, diagonally upward and diagonally downward in either the right or left directions, or combinations thereof, while the user's eye remains closed. The user may be asked to perform multiple particular eye positions sequentially.

At 1303, the method includes receiving, with one or more photodetectors 144, electromagnetic radiation reflected from the closed eye of the user performing the particular eye position(s), either individually or in sequence. In some embodiments, electromagnetic radiation emitted by the emitters 142 reflects off the eyelid and underlying ocular geometry, and the reflected electromagnetic radiation is received by one or more photodetectors 144. The reflected radiation may vary spatially and in intensity based on contour variations of the eyelid caused by the position of the eye beneath the eyelid.

At 1304, the method includes outputting, from the photodetectors 144, an eye position signal to the computing device and processor. The photodetectors 144 may generate one or more electrical signals indicative of the received electromagnetic radiation. These signals may be output to the computing device 102 and processor 712 as an eye position signal representing the reflected electromagnetic radiation associated with the particular eye position of the user.

At 1305, the method includes producing, by the processor 712, a spatial intensity pattern based on the eye position signal. In some embodiments, the processor 712 analyzes the eye position signal to generate a spatial intensity pattern representing a distribution of reflected electromagnetic radiation across one or more sensing regions. The spatial intensity pattern may correspond to intensity variations caused by the geometry of the eyelid and the dome-shaped cornea beneath the eyelid for the particular eye position.

At 1306, the method includes storing the spatial intensity pattern in computer-readable memory in memory device(s) 720. The spatial intensity pattern 901 may be stored in memory associated with the computing device 102 as calibration data corresponding to the specific user and the particular eye position. In some embodiments, multiple spatial intensity patterns may be generated and stored for different eye positions, thereby creating a user-specific calibration profile for detecting eye positions of the user when using sleep mask device 100.

The calibration method 1300 may be repeated for multiple eye positions to generate a set of spatial intensity patterns associated with the user. During subsequent operation of the sleep mask device, real-time spatial intensity patterns may be compared to the stored calibration patterns to determine eye position, detect eye movement, or map detected eye positions to operational commands that trigger operations of the sleep mask device 100.

Although the steps of method 1300 are illustrated in a particular order, the steps may be performed in different orders, repeated, combined, or omitted in various embodiments without departing from the scope of the present disclosure. Additionally, one or more steps of method 1300 may be performed automatically, without explicit user prompts, or during normal use of the sleep mask device.

### Sleep Mask Device Control and Operation Based on Eye Position Detection

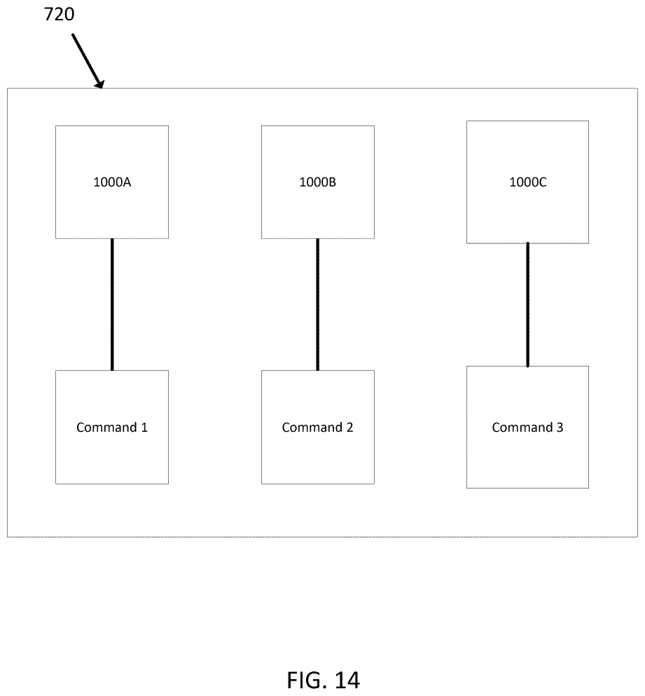

As recited above, sleep mask device 100 can detect eye position and eye motion of eyes of a user who is using sleep mask device 100. Sleep mask device 100 can further utilize this detection of eye position and motion to perform functions and operations with sleep mask device 100. As shown in FIG. 14, a plurality of eye positions can be stored in the memory devices 720 in connection with a function of sleep mask device 100. In other words, eye positions and/or eye movements, performed by a user with their eyes closed, can be stored in memory device(s) 720 in connection with instructions stored in non-transitory computer-readable storage media that, when executed by the processor 712, performs a predefined function or operational command of the sleep mask device 100, which operational command is associated with the stored instructions to trigger a function of the sleep mask device 100. For example, as shown in FIG. 14, eye position 1000A from FIG. 10 can be associated with instructions stored in memory device 720 to perform an operational command, specifically Command 1. Similarly, eye position 1000B from FIG. 10 can be associated with instructions stored in memory device 720 to perform an operational command, specifically, Command 2, and eye position 1000C from FIG. 10 can be associated with instructions stored in memory device 720 to perform an operational command, specifically, Command 3.

While not shown, any other eye positions capable of being performed by a user can also be associated with an operational command of the sleep mask device 100. Any number of eye positions that are performable by a user with their eyes closed can be associated with commands to trigger, alter, or stop operations of the sleep mask device 100. For example, a gaze-to-command module can be included in modules 724. This module can generate discrete or continuous operational commands based on detected eye position of the closed-eye of the user. In various embodiments, an operational command can be triggered based on one or more of the following detected eye-related parameters: absolute eye position within a defined spatial coordinate system; relative eye position with respect to a baseline or reference position; directional eye movement (e.g., leftward, rightward, upward, downward); magnitude of eye movement; velocity or acceleration of eye movement; dwell time of the eye at a particular position or within a defined region; repetitive or oscillatory eye movements; sequential eye movements occurring in a defined temporal order; blink detection, blink rate, or blink duration; combinations of any of the foregoing. When the eye position or motion is detected by the processor 712, the processor 712 can then execute the instructions and operational command(s) associated with the detected eye position based on the association stored in memory device(s) 720 between the detected eye position of the user.

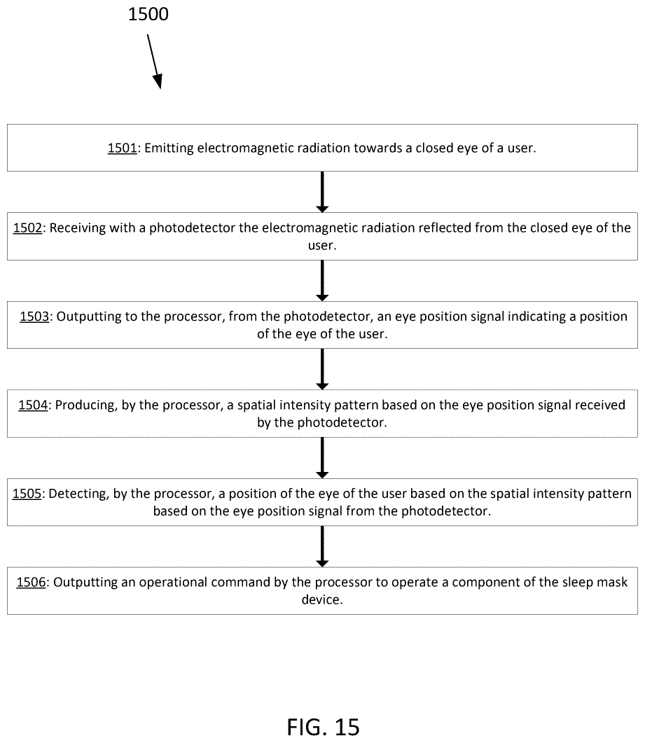

Referring now to FIG. 15, a method 1500 is shown for detecting an eye position of a user and operating a component or executing an operational command of the sleep mask device 100 based on the detected eye position. In some embodiments, the method 1500 is performed using user-specific calibration data generated according to the calibration method 1300 described above with reference to FIG. 13.

At step 1501, the method 1500 includes emitting electromagnetic radiation toward a closed eye of a user. One or more emitters 142 of the sleep mask device 100 may emit electromagnetic radiation toward a closed eyelid of the user while the sleep mask device 100 is worn. The electromagnetic radiation may include infrared or near-infrared radiation and may illuminate at least a portion of the eyelid and underlying ocular structures.

At step 1502, the method 1500 includes receiving, with one or more photodetectors 144, electromagnetic radiation reflected from the closed eye of the user. The electromagnetic radiation emitted by the emitters 142 may reflect off the eyelid and underlying ocular geometry, and the reflected electromagnetic radiation may be received by one or more photodetectors 144.

At step 1503, the method 1500 includes outputting, from the photodetector to the processor 712, an eye position signal indicating a position of the eye of the user. The photodetectors 144 may generate one or more electrical signals corresponding to the received electromagnetic radiation, and the signals may be provided to the computing device 102 and processor 712 as an eye position signal.

At step 1504, the method 1500 includes producing, by the processor 712, a spatial intensity pattern based on the eye position signal received by the photodetector. In some embodiments, the processor 712 analyzes the eye position signal to generate a spatial intensity pattern representing a spatial distribution of reflected electromagnetic radiation across one or more sensing regions. The spatial intensity pattern may be influenced by contour variations of the eyelid caused by the position of the eye beneath the eyelid.

At step 1505, the method 1500 includes detecting, by the processor 712, a position of the eye of the user based on the spatial intensity pattern. In some embodiments, the processor 712 compares the spatial intensity pattern produced at step 1504 to one or more stored spatial intensity patterns generated during a prior calibration process for the user. Based on the comparison, the processor 712 may determine a current eye position of the user, such as a directional gaze or predefined eye gesture, while the eye remains closed.

At step 1506, the method includes outputting an operational command by the processor 102 to operate a component of the sleep mask device 100. The operational command may be mapped to the detected eye position and may control one or more components of the sleep mask device 100, including but not limited to audio output, initiation or modification of a sleep program, volume adjustment, playback control, or other device functions.

In some embodiments, method 1500 is performed continuously or repeatedly during use of the sleep mask device 100 to enable real-time eye-based control. The method may further include updating stored spatial intensity patterns over time to adapt to changes in user behavior, device positioning, or physiological conditions.

Although the steps of method 1500 are illustrated in a particular order, the steps may be performed in different orders, repeated, combined, or omitted in various embodiments without departing from the scope of the present disclosure. Additionally, one or more steps may be performed automatically without user awareness or explicit prompts.

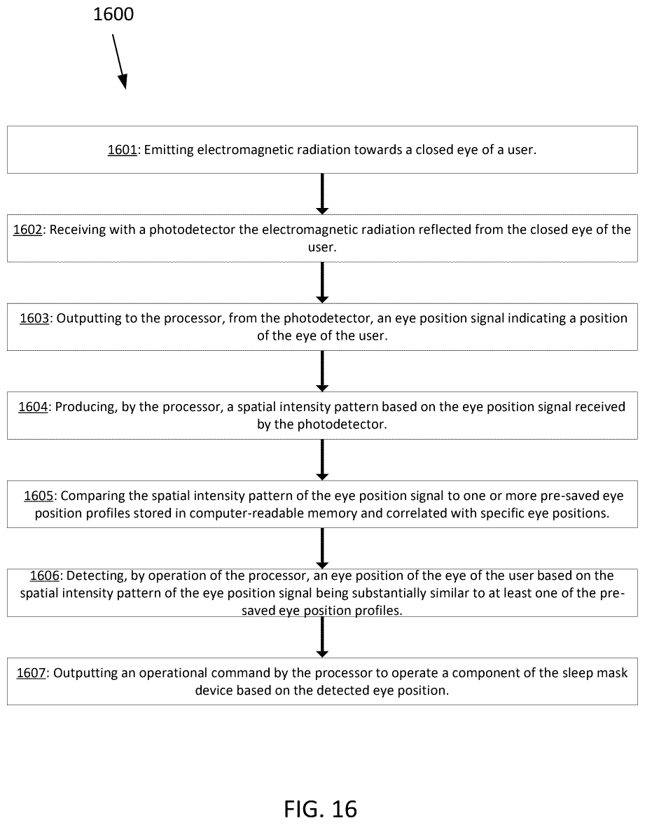

The method 1500 can be altered and built upon to create more detailed or different methods. For example, referring now to FIG. 16, a method 1600 is shown for detecting an eye position of a user based on comparison of a spatial intensity pattern to one or more pre-saved eye position profiles, and for operating a component of the sleep mask device 100 based on the detected eye position. In some embodiments, the method 1600 is performed using calibration data generated according to the calibration methods described above with reference to FIG. 13.

At step 1601, the method 1600 includes emitting electromagnetic radiation toward a closed eye of a user. One or more emitters 142 of the sleep mask device 100 emit electromagnetic radiation toward a closed eyelid of the user while the sleep mask device 100 is worn. The emitted electromagnetic radiation may include infrared or near-infrared radiation and may illuminate the eyelid and underlying ocular structures.

At step 1602, the method 1600 includes receiving, with one or more photodetectors 144, electromagnetic radiation reflected from the closed eye of the user. The reflected electromagnetic radiation may vary based on the geometry of the eyelid and the position of the eye or cornea beneath the eyelid, and may be received by one or more photodetectors 144 positioned within the sleep mask device 100.

At step 1603, the method 1600 includes outputting, from the photodetector to the processor 712, an eye position signal indicating a position of the eye of the user. The photodetectors 144 generate one or more electrical signals corresponding to the received electromagnetic radiation, and the signals are provided to the computing device 102 and processor 712 as an eye position signal.

At step 1604, the method includes producing, by the processor 712, a spatial intensity pattern based on the eye position signal. The processor 712 analyzes the eye position signal to generate a spatial intensity pattern representing a spatial distribution of reflected electromagnetic radiation across one or more sensing regions. The spatial intensity pattern may correspond to contour variations of the eyelid caused by the position of the eye beneath the eyelid.

At step 1605, the method 1600 includes comparing the spatial intensity pattern to one or more pre-saved eye position profiles stored in computer-readable memory and correlated with specific eye positions. Each pre-saved eye position profile may represent a spatial intensity pattern previously generated during a calibration process for the user and associated with a known eye position or eye gesture.

At 1606, the method 1600 includes detecting, by operation of the processor 712, an eye position of the eye of the user based on the spatial intensity pattern being substantially similar to at least one of the pre-saved eye position profiles. In some embodiments, the processor 712 determines similarity based on one or more criteria, such as intensity distribution, relative intensity differences, pattern shape, correlation scores, threshold comparisons, or combinations thereof.

At step 1607, the method 1600 includes outputting an operational command by the processor 712 to operate a component of the sleep mask device 100 based on the detected eye position. The operational command may be mapped to the detected eye position and may control one or more components of the sleep mask device 100, including audio output, initiation or modification of a sleep program, volume adjustment, playback control, communication with an external device, or other device functions.

In some embodiments, the method 1600 is performed repeatedly or continuously during use of the sleep mask device, thereby enabling real-time or near-real-time eye-based control while the user's eyes remain closed. In some embodiments, the pre-saved eye position profiles may be updated, refined, or supplemented over time to adapt to changes in user behavior, device positioning, or physiological conditions.

Although the steps of method 1600 are illustrated in a particular order, the steps may be performed in different orders, repeated, combined, or omitted in various embodiments without departing from the scope of the present disclosure.

Several examples of operational commands and associated eye positions, movements, and speeds will be described herein. As a first example of performing an operational command (e.g., command 1, 2, or 3), a user wearing the sleep mask device 100 can adopt a particular eye gaze position to perform an operational command of the sleep mask device 100. A user may move their closed eye to a first position (e.g., an upward position), which first position can be associated with an operational command of powering off the sleep mask device 100. By adopting the first eye position, the user controls the sleep mask device to turn off. Other similar operations can be mapped to a multitude of functions and operational commands to offer closed eye operability of the sleep mask device 100. A user may adopt a second eye position to perform an operational command to turn the volume of speakers 150 up. A user may adopt a third eye position to perform and operational command to turn the volume of speakers 150 down.

Similarly, the user can perform an eye motion to perform similar operational commands and functions of the sleep mask device 100. For example, a user can perform a first motion in a first direction (e.g., moving the eyes from a downward gazing position to an upward gazing position) in order to perform an operational command to turn the volume up. A user can perform a second motion (e.g., moving the eyes from an upward gazing position to a downward gazing position) in order to perform an operational command to turn the volume down. As another example, the speed of the motion can also factor into the first and second motion of the eyes and control which function is performed based on the velocity of the eye movement. For example a user can perform a first motion in a first direction (e.g., moving the eyes from a downward gazing position to an upward gazing position at a predefined velocity) in order to perform an operational command to turn the volume up. A user can perform a second motion (e.g., moving the eyes from an upward gazing position to a downward gazing position at a predefined velocity) in order to perform an operational command to turn the volume down.

Any number of functions and operational commands can be performed based on eye position, eye movement direction, and eye movement speed/acceleration. As some examples of operational commands, the sleep mask device can be muted, turned off, volume turned up, volume turned down, exiting programs being run on the sleep mask device, initiating programs to be run on sleep mask device, turning on/off microphone 160, and disconnecting or connecting to an external device (e.g., pairing).

The detected eye position or movement may be mapped to an operational command using a lookup table, rule-based logic, threshold comparison, machine-learned model, or adaptive algorithm. In some embodiments, detected eye movements may be used to control power or operational states of the electronic sleep mask device, including but not limited to: powering the device on or off; transitioning between active, standby, and sleep modes; initiating or terminating an audio output; locking or unlocking user input controls; waking the device from a low-power state. For example, a sustained upward gaze maintained for a threshold dwell time may initiate a power-on command, while a sustained downward gaze may initiate a power-off or standby command.

In some embodiments, eye-based control commands may be used to navigate among selectable options, content, or menus associated with the device, including but not limited to: selecting or deselecting a therapy/sleep program having predefined audio and guided eye movements; navigating between menu items or settings; confirming or canceling a selection; advancing to a next option or returning to a previous option. For example, lateral eye movements may be interpreted as navigation commands, while maintaining eye position at a selected location for a defined dwell time may be interpreted as a selection or confirmation command.

In some embodiments, detected eye position or movement may be used to adjust one or more therapy/sleep program parameters provided by the electronic sleep mask device, including but not limited to: intensity, brightness, or wavelength of emitted light; audio volume, frequency, or pattern; vibration intensity, duration, or frequency of haptic feedback; timing, duty cycle, or sequencing of therapeutic stimuli such as audio or directions provided to a user. For example, upward eye movement may increase an audio volume level, while downward eye movement may decrease the audio volume level.

In some embodiments, eye-based control commands may be used to control timing-related functions, including but not limited to: starting, pausing, or stopping a sleep or relaxation session; delaying initiation of therapy by a predetermined period; snoozing or dismissing an alarm or wake-up stimulus; adjusting timing based on detected sleep stage or circadian phase. For example, a repetitive eye movement pattern detected during a wake-up phase may be interpreted as a snooze command for an audio or haptic alarm used to wake a user.

In some embodiments, eye-based control commands may be used to initiate or modify calibration or personalization processes, including but not limited to: initiating an eye-tracking calibration routine; storing a baseline eye position for a user; selecting among user profiles; adapting control sensitivity based on detected eye behavior. For example, a predefined sequence of eye movements may initiate a calibration process that updates mapping between detected eye position and control commands.

In some embodiments, the mapping between detected eye position or movement and control commands may be fixed, user-configurable, or dynamically adapted over time. The device may update control mappings based on historical eye movement data, user feedback, detected fatigue, or changes in sensor performance. In certain embodiments, multiple detected eye movement parameters may be combined to reduce false triggering, such as requiring both a directional movement and a minimum dwell time before executing a control command. In some embodiments, the electronic sleep mask device may support hierarchical or multi-stage control commands, wherein an initial eye movement places the device into a command-selection mode and subsequent eye movements select and execute a specific command. For example, a first detected eye movement may activate a control mode, and one or more subsequent detected eye movements within a defined time window may specify a particular device function to be controlled.

Unless otherwise indicated, references to “operational commands” generated based on detected eye position or movement shall be understood to include commands generated based on single eye measurements, combined measurements from both eyes, temporal sequences of measurements, or statistical representations of eye movement data over time.

### Sleep Inducing Programs and Audio Output Based on Detected Eye Position

The sleep mask device 100 can help to facilitate a user falling to sleep by occluding the user's vision, blocking out external light, and by providing sleep inducing programs for the user to participate in which are meant to induce sleep. As an aspect of the sleep-inducing programs, audio sounds and feedback are provided to the user through speakers 150, or an external speaker 1204 to allow the user to interact with stimuli without needing to open their eyes. In order to better facilitate a user falling to sleep, the sleep mask device 100, as described herein, allows for closed eye detection of eye position and movement to put a user in a more natural sleep state as they perform tasks or participate in programs that will help them fall asleep. As open eyes are not conducive to sleeping, the closed eye position and motion detection is beneficial to help a user fall asleep.

As a further example of an operational command performed by the processor 712 of the sleep mask device 100 in response to detected eye positions, as well as movement directions and velocities/accelerations, the processor 712 may perform an operational command to output an audio signal to cause one or more speakers 150 to produce audio sounds that are based on the detected position or movement of the user's eyes. This disclosure illustrates several examples of how the eye tracker sensor 140 can detect eye position, movement direction, and movement speed/acceleration of a closed eye through analysis of reflected electromagnetic radiation, enabling control of audio output in accordance with eye position or movement. This detection of eye position and movement can be used to engage a user in a sleep-inducing program while triggering audio feedback, sounds, narrative passages, audio-based spatial environments and more to lull a user to sleep. It is to be understood that any operational commands or eye gaze patterns, positions, or motions described anywhere in this disclosure can also be applicable to control of audio output by the sleep mask device 100.

Several examples of sleep-inducing programs can be run on sleep mask device 100 to help lull a user to sleep. The sleep-inducing programs can include operational commands executed by the processor 712 to the cause the speaker(s) 150 to output an audio sound, alter an audio sound, or cease an audio sound based on the eye position signal output by the eye tracker sensor 140 and indicating the position of the eye detected by the processor 712.

Examples of sleep-inducing programs include the following. In some embodiments, discrete eye positions can be mapped to distinct audio outputs. For example, a first position such as “left” causes the processor 171 to output a first audio signal that results in a first musical pitch or a first narrative passage, and a second position such as “right” causes output of a second, different pitch or a second narrative passage.

In some embodiments, detected movement direction of the eye of a user modulates an ongoing audio stream. For example, an upward movement can increase pitch or add a brightening modulation and a downward movement can decrease pitch or apply a damping modulation.

In some embodiments, a narrative module of modules 724 can output a sequence of audio passages and allows a user to select subsequent passages based on detected eye position or movement at certain points within the narrative. If a user does not make a choice after a certain threshold, the narrative may continue on a default path.

In some embodiments, a melody subsystem module of modules 724 can map eye positions to musical notes or chord selection and maps eye movement and direction to modulation of pitch, amplitude, or timbre over time. The melody can be generated procedurally or retrieved from stored patterns in memory devices 720 and can be simplified automatically as the user approaches sleep.

In some embodiments, a task subsystem module of modules 724 can provide audio instructions for simple routines such as breath pacing, and treats predetermined eye positions, movements, and directions as status inputs. Upon detection of an input, the task subsystem module can provide an acknowledgement sound and proceed to the next instruction. Parameters such as tempo and volume can adapt to measured responsiveness of the user.

In some embodiments, the spatial-audio engine modules of modules 724 can create a virtual sound environment whose orientation can be adjusted based on eye position, direction, and motion detection or input. The system can simulate objects at specified azimuth and elevation angles and can trigger selection or interaction sounds when the user directs their eye gaze toward a perceived object location in the spatial-audio environment. These features can be used alone or in combination with narrative and melody features.

In one example mode, a game module of modules 724 can be included in which an audio game is initiated in which a sheep sound moves from left to right in the perceived scene and the user confirms presence of a sheep by looking in a pre-defined first direction. A confirmation chime can indicate correct selection. When a non-sheep sound such as a pig occurs, the user looks in a pre-defined second direction to register a “no.”

The sleep mask device 100 can include a sleep detection module in modules 724, that operates such that, when a user is detected to move towards sleep based on input from the sleep mask device 100 or activity tracker 1202, the pace of events and audio level in any of the above examples can automatically reduce as detected eye activity slows, transitioning toward silence when the user falls asleep.

Specific general examples of starting, stopping, or altering audio output based on detected eye position, eye movement, eye velocity, spatial intensity patterns, and/or eye position signals are given below. The processor 712 of the sleep mask device 100 can output a first audio signal to the one or more speakers 150 causing the one or more speakers to output a first audio sound when the eye position signal from the eye tracker sensor 140 indicates the position of the one or more eyes of the user is at a first position (e.g., up, down, right, left, etc.). The processor 712 can output a second audio signal to the one or more speakers 150 causing the one or more speakers to output a second audio sound different from the first audio sound when the eye position signal from the eye tracker sensor 140 indicates the position of the one or more eyes of the user is at a second position different than the first position (e.g., up, down, right, left, etc.). The first audio sound can be a first musical pitch (e.g., a C note) and the second audio sound can be a second musical pitch (e.g., an A note) different from the first musical pitch.

Additionally or alternatively, the processor 712 can output a first audio signal to the one or more speakers 150 causing the one or more speakers to output a first audio sound when the eye tracker sensor 140 detects motion of the one or more eyes of the user in a first direction. The processor 712 can further output a second audio signal to the one or more speakers 150 causing the one or more speakers 150 to output a second audio sound different from the first audio sound when the eye tracker sensor detects motion of the one or more eyes of the user in a second direction different from the first direction.

The processor 712 can output a third audio signal to the one or more speakers 150 causing the one or more speakers 150 to output a third audio sound (e.g, upward modulation in pitch, downward modulation in pitch, increase in volume, decrease in volume, etc.) when the eye position signal from the eye tracker sensor 140 indicates motion of the one or more eyes of the user in a first direction (e.g., upward moving direction, downward moving direction, left moving direction, right moving direction, etc.). The processor 712 can output a fourth audio signal to the one or more speakers 150 causing the one or more speakers 150 to output a fourth audio sound (e.g, upward modulation in pitch, downward modulation in pitch, increase in volume, decrease in volume, etc.) different from the third audio sound when the eye position signal from the eye tracker sensor 140 indicates motion of the one or more eyes of the user in a second direction different from the first direction. The third audio sound and the fourth audio sound can each be modulations of one or more of the first audio sound and the second audio sound, for example, the modulations can be an increase in pitch or a decrease in pitch of the first and second audio sounds, or a change in tone quality or timbre. The change in the first and second audio sounds indicated by the third and fourth audio sounds is not intended to be particularly limited by this disclosure.

In cases in which a user is listening to a narrative passage on the sleep mask device 100, the processor 712 outputs audio signals to the one or more speakers 150 causing the one or more speakers 150 to output audio sounds of a narrative to the user. In this case, the first audio sound can be a first narrative passage and the second audio sound can be a second narrative passage different from the first narrative passage. The user can choose the narrative passage to follow by inputting eye movements while listening to the narrative passage in order to alter the story the user is hearing. The processor 712 can output audio signals to the one or more speakers to alter the narrative based on one or more of the position and motion of the one or more eyes of the user beneath the eyelid detected based on the eye position signal output by the eye tracker sensor 140 and detected based on the analysis of the processor performed on the eye position signal.

The processor 712 can further output audio signals to the one or more speakers 150 causing the one or more speakers 150 to output audio sounds of a melody to the user. The processor 712 can output audio signals to the one or more speakers to alter the melody based on one or more of the position and motion of the one or more eyes of the user beneath the eyelid detected based on the eye position signal output by the eye tracker sensor 140 and detected based on the analysis of the processor performed on the eye position signal.

The processor 712 can further output audio signals to the one or more speakers 150 causing the one or more speakers 150 to output audio sounds of instructions to perform tasks to the user. The tasks are tasks performed exclusively by motion of the user's eyes while the eyes are closed in order to create an environment conducive to sleep. The processor 712 can output audio signals to the one or more speakers to indicate a status of the tasks being performed by the user based on one or more of the position and movement of the one or more eyes of the user beneath the eyelid detected based on an eye position detected based on the eye position signal output by the eye tracker sensor 140 and detected based on the eye position signal output by the eye tracker sensor 140 and detected based on the analysis of the processor performed on the eye position signal.

The processor 712 can further output audio signals to the one or more speakers 150 causing the one or more speakers 150 to output audio sounds to create a spatial audio environment for the user. The processor 712 can output audio signals to the one or more speakers to alter orientation of the spatial audio environment based on one or more of the position and movement of the one or more eyes of the user beneath the eyelid detected based on the eye position signal output by the eye tracker sensor 140 and detected based on the analysis of the processor performed on the eye position signal. The user can navigate the spatial audio environment by moving their closed eyes. The audio sounds will be perceived to move in stereo around the user as the user shifts their gaze around the spatial audio environment.

The processor 712 can further output audio signals to the one or more speakers 150 causing the one or more speakers 150 to output audio sounds simulating the presence of one or more simulated objects in the spatial audio environment. The processor 712 can output audio signals to the one or more speakers 150 to simulate user interaction with the one or more simulated objects in the spatial audio environment based on one or more of the position and movement of the one or more eyes of the user beneath the eyelid detected based on the eye position signal output by the eye tracker sensor 140 and detected based on the analysis of the processor performed on the eye position signal.

### Example Workflow Process

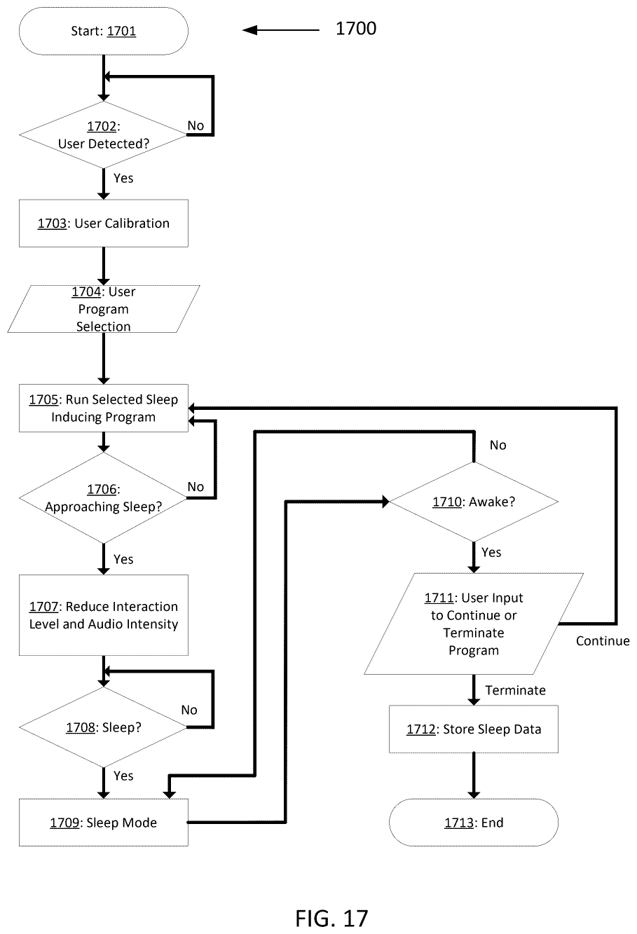

Referring now to FIG. 17, an exemplary workflow method 1700 is illustrated for operating the sleep mask device to guide a user through calibration, sleep induction, sleep monitoring, and post-sleep handling. The method 1700 may be executed by the computing device 102 and processor 712 of the sleep mask device described herein, based on sensor data obtained from one or more sensors of the device.

The method begins at 1701, in which the user wears and turn on the device. At 1702, the processor 712 determines whether a user is detected wearing sleep mask device 100. User detection may be based on one or more criteria, including but not limited to detection of the sleep mask being worn, detection of eye-related signals from an eye tracker sensor, detection of motion, biometric data, or combinations thereof. If a user is not detected, the method may loop until a user is detected.

If a user is detected at step 1702, the method proceeds to 1703, where user calibration is performed. User calibration may include executing one or more calibration routines, such as the calibration methods described above with reference to FIG. 13, to generate or confirm user-specific eye position profiles or spatial intensity patterns.

At 1704, the method includes user program selection. In some embodiments, the processor 712 receives user input to select a sleep-inducing program, such as a particular audio program, narrative, soundscape, or other sleep assistance routine. The selection may be received via eye-based input, an external device, or another input modality.

At 1705, the processor 712 runs the selected sleep-inducing program. Running the sleep-inducing program may include outputting audio via one or more speakers 150, adjusting audio parameters over time, or performing other operations intended to facilitate relaxation or sleep onset.

At 1706, the processor 712 determines whether the user is approaching sleep. This determination may be based on one or more indicators, including eye activity, eye movement patterns, reduced interaction frequency, biometric data, or other sensor-derived information, including biometric data from a smart watch, fitness tracker, or other wearable activity tracker device. If the user is not approaching sleep, the method may return to step 1705 and continue running the selected sleep-inducing program until the user terminates the program or falls asleep.

If the processor 712 determines that the user is approaching sleep at step 1706, the method proceeds to 1707, where the processor reduces interaction level and audio intensity of the sleep-inducing program. Reducing interaction level may include decreasing the frequency or sensitivity of eye-based input detection, while reducing audio intensity may include lowering volume, simplifying audio content, or transitioning to more passive audio output.

At 1708, the processor 712 determines whether the user is asleep. If the user is not yet asleep, the method may loop to continue reduced-interaction operation until sleep is detected. If sleep is detected, the method proceeds to 1709, where the device enters a sleep mode. In sleep mode, the processor 712 may minimize active outputs, conserve power, and continue passive monitoring of the user.

At 1710, the processor 712 determines whether the user is awake. If the user is not awake, the method may continue operating in sleep mode. If the user is determined to be awake, the method proceeds to 1711, where user input to continue or terminate the program is received. The user input may be detected via eye-based input, motion, or another input mechanism.

If the user elects to continue, the method may return to step 1705 to resume or adjust the sleep-inducing program. If the user elects to terminate the program, the method proceeds to 1712, where the processor stores sleep data. Stored sleep data may include sleep duration, sleep stages, eye activity metrics, program usage data, or other information associated with the sleep session. The method concludes at 1713.

Although the steps of method 1700 are illustrated in a particular order, the steps may be performed in different orders, repeated, combined, or omitted in various embodiments without departing from the scope of the present disclosure. Additionally, one or more steps may be performed automatically by the processor 712 without explicit user awareness.

Although specific embodiments, implementations, and examples have been described herein for purposes of illustration, it will be appreciated that the present disclosure is not limited to those embodiments. Various modifications, substitutions, rearrangements, and combinations of the disclosed features may be made without departing from the spirit and scope of the disclosure. The described components, modules, and methods may be implemented in hardware, software, firmware, or any combination thereof, and functionality described with respect to one embodiment may be incorporated into other embodiments. Additionally, the order of operations described in the methods illustrated herein is not intended to be limiting, and the steps may be performed in different orders, concurrently, repeated, combined, or omitted in certain implementations. None of the described operations should be interpreted as requiring human mental steps, and all determinations described herein may be performed by electronic processing circuitry. Accordingly, the scope of the present disclosure is not limited by the specific embodiments described herein, but is instead defined by the claims and their equivalents.

What is claimed is:

1. A head-mounted electronic sleep mask device comprising:
a support portion configured to secure the sleep mask device to a head of a user when in use;
a mask portion connected to the support portion and configured to be placed over one or more eyes of a user to occlude vision of the user when in use;
an eye tracker sensor supported on the mask portion and configured to output an eye position signal indicating an eye position of one or more eyes of the user;
a processor in electronic communication with the eye tracker sensor and the speaker; and
a non-transitory computer-readable storage medium that stores instructions that, when executed by the processor, executes an operational command of the head-mounted electronic sleep mask device;
wherein the operational command executed by the processor based on the instructions is based on an eye position detected based on the eye position signal output by the eye tracker sensor.

2. The head-mounted electronic sleep mask device of claim 1, wherein the eye tracker sensor is configured to produce the eye position signal, indicating the eye position of the one or more eyes, when the one or more eyes are closed based on the contour of an eyelid covering the eye.

3. The head-mounted electronic sleep mask device of claim 2, wherein the eye tracker sensor comprises:
one or more emitters configured to emit electromagnetic radiation; and
one or more photodetectors configured to convert the electromagnetic radiation emitted by the emitters to the eye position signal.

4. The head-mounted electronic sleep mask device of claim 3 wherein, when in use, the electromagnetic radiation from the one or more emitters is incident on a portion of the eyelid covering the eye and the one or more photodetectors receives the electromagnetic radiation from the one or more emitters and converts the electromagnetic radiation to the eye position signal indicating a position of one or more eyes of the user based on the contour of the eyelid covering the eye of the user.

5. The head-mounted electronic sleep mask device of claim 4 wherein, when in use, the electromagnetic radiation from the one or more emitters is incident on a portion of the eyelid covering the eye and the one or more photodetectors receive reflected electromagnetic radiation from the external surface of the eyelid and produce the eye position signal, indicating the position of one or more eyes of the user, based on one or more of spatial intensity variations in the reflected electromagnetic radiation from the external surface of the eyelid, wherein the spatial intensity variations correlate with eye positions of the one or more eyes of the user.

6. The head-mounted electronic sleep mask device of claim 5, further comprising:
a speaker configured to output audio;
wherein the operational command executed by the processor is to the cause the speaker to output an audio sound based on the eye position signal output by the eye tracker sensor and indicating the position of the eye, and
wherein the processor outputs a first audio signal to the one or more speakers causing the one or more speakers to output a first audio sound when the eye position signal from the eye tracker sensor indicates the position of the one or more eyes of the user is at a first position.

7. The head-mounted electronic sleep mask device of claim 6, wherein the processor outputs a second audio signal to the one or more speakers causing the one or more speakers to output a second audio sound different from the first audio sound when the eye position signal from the eye tracker sensor indicates the position of the one or more eyes of the user is at a second position different than the first position.

8. The head-mounted electronic sleep mask device of claim 7, wherein the processor outputs a third audio signal to the one or more speakers causing the one or more speakers to output a third audio sound when the eye position signal from the eye tracker sensor indicates motion of the one or more eyes of the user in a first direction.

9. The head-mounted electronic sleep mask device of claim 8, wherein the processor outputs a fourth audio signal to the one or more speakers causing the one or more speakers to output a fourth audio sound different from the third audio sound when the eye position signal from the eye tracker sensor indicates motion of the one or more eyes of the user in a second direction different from the first direction.

10. The head-mounted electronic sleep mask device of claim 7, wherein the first audio sound is a first musical pitch and the second audio sound is a second musical pitch different from the first musical pitch.

11. The head-mounted electronic sleep mask device of claim 9, wherein the third audio sound and the fourth audio sound are each modulations of one or more of the first audio sound and the second audio sound.

12. The head-mounted electronic sleep mask device of claim 7, wherein the first audio sound is a first narrative passage and the second audio sound is a second narrative passage different from the first narrative passage.

13. The head-mounted electronic sleep mask device of claim 5, wherein, when in use, the processor outputs audio signals to the one or more speakers causing the one or more speakers to output audio sounds of a narrative to the user; and
wherein the processor outputs audio signals to the one or more speakers to alter the narrative based on one or more of the position and motion of the one or more eyes of the user beneath the eyelid detected based on the eye position signal output by the eye tracker sensor.

14. The head-mounted electronic sleep mask device of claim 5, wherein, when in use, the processor outputs audio signals to the one or more speakers causing the one or more speakers to output audio sounds of a melody to the user; and
wherein the processor outputs audio signals to the one or more speakers to alter the melody based on one or more of the position and motion of the one or more eyes of the user beneath the eyelid detected based on the eye position signal output by the eye tracker sensor.

15. The head-mounted electronic sleep mask device of claim 5, wherein, when in use, the processor outputs audio signals to the one or more speakers causing the one or more speakers to output audio sounds of instructions to perform tasks to the user; and
wherein the processor outputs audio signals to the one or more speakers to indicate a status of the tasks being performed by the user based on one or more of the position and movement of the one or more eyes of the user beneath the eyelid detected based on the eye position signal output by the eye tracker sensor.

16. The head-mounted electronic sleep mask device of claim 5, wherein, when in use, the processor outputs audio signals to the one or more speakers causing the one or more speakers to output audio sounds to create a spatial audio environment; and
wherein the processor outputs audio signals to the one or more speakers to alter orientation of the spatial audio environment based on one or more of the position and movement of the one or more eyes of the user beneath the eyelid detected based on the eye position signal output by the eye tracker sensor.

17. The head-mounted electronic sleep mask device of claim 16, wherein, when in use, the processor outputs audio signals to the one or more speakers causing the one or more speakers to output audio sounds simulating the presence of one or more simulated objects in the spatial audio environment; and
wherein the processor outputs audio signals to the one or more speakers to simulate user interaction with the one or more simulated objects in the spatial audio environment based on one or more of the position and movement of the one or more eyes of the user beneath the eyelid detected based on the eye position signal output by the eye tracker sensor.

18. The head-mounted electronic sleep mask device of claim 5, wherein the processor outputs a first audio signal to the one or more speakers causing the one or more speakers to output a first audio sound when the eye tracker sensor detects motion of the one or more eyes of the user in a first direction.

19. The head-mounted electronic sleep mask device of claim 18, wherein the processor outputs a second audio signal to the one or more speakers causing the one or more speakers to output a second audio sound different from the first audio sound when the eye tracker sensor detects motion of the one or more eyes of the user in a second direction different from the first direction.

20. A head-mounted electronic sleep mask device comprising:
a support portion configured to secure the sleep mask device to a head of a user when in use;
a mask portion connected to the support portion and configured to be placed over eyes of a user to occlude vision of the user when in use;
an eye tracker sensor supported on the mask portion and configured to produce an eye position signal indicating an eye position of one or more eyes of the user;
a processor in electronic communication with the eye tracker sensor; and
a non-transitory computer-readable storage medium that stores instructions that, when executed by the processor, outputs a signal configured to cause an external speaker to output an audio sound;
wherein the signal output by the processor is based on the eye position signal output by the eye tracker sensor.
